(19) United States
(12) Patent Application Publication
(10) Pub. No.: US 2024/0200022 A1
(43) Pub. Date: Jun. 20, 2024

(54) METHODS AND SYSTEMS FOR THE CULTURE OF CELLS AT LIQUID-LIQUID INTERFACES

(71) Applicant: QUEEN MARY UNIVERSITY OF LONDON, London (GB)

(72) Inventors: Julien Gautrot, London (GB); Dexu Kong, London (GB); Yaqi You, London (GB); Lihui Peng, London (GB)

(21) Appl. No.: 18/505,438

(22) Filed: Nov. 9, 2023

Related U.S. Application Data
(63) Continuation of application No. 16/958,862, filed on Jun. 29, 2020, now abandoned.

(30) Foreign Application Priority Data
Dec. 28, 2017 (GB) 1722186.2

(51) Int. Cl. C12N 5/00; C12N 5/071; C12N 5/074

(52) U.S. Cl. CPC C12N 5/0068; C12N 5/0629; C12N 5/0696; C12N 2533/30; C12N 2533/32; C12N 2533/50; C12N 2533/52; C12N 2533/54

(57) ABSTRACT

The present invention relates to methods of culturing adherent cells, in particular adherent stem cells, to confluency at a liquid-liquid interface. The invention also provides cell culture systems useful in the culture of cells at liquid-liquid interfaces. The cell culture systems generally comprise an aqueous cell culture medium and an oil phase, there being a conditioning layer disposed between the cell culture medium and the oil phase comprising a peptide or polymer layer and a surfactant that assists in the culture of the adherent cells (in particular stem cells) at the interface between the two phases.

The present invention relates to methods of culturing adherent cells, in particular adherent stem cells, at a liquid-liquid interface. The invention also provides cell culture systems useful in the culture of cells at liquid-liquid interfaces. The cell culture systems generally comprise an aqueous cell culture medium and an oil phase, there being a conditioning layer disposed between the cell culture medium and the oil phase comprising a peptide or polymer layer and a surfactant that assists in the culture of the adherent cells (in particular stem cells) at the interface between the two phases.

The cell culture systems provide a substrate of sufficient rigidity and viscoelasticity to allow the culture of cells to confluency. The present invention is also proposed to allow the culture of adherent cells at liquid-liquid interfaces over a longer period that previously possible in methods of the art.

### BACKGROUND

Substrate mechanics and topography play an important role in regulating biochemical signals such as integrin-mediated matrix anchorage and cell spreading (Di Cio, S. & Gautrot, J. E., Acta Biomater 30, 26-48, 2016). Such physical cues have a striking impact on cell phenotype, such as the differentiation of stem cells and the preservation of their potency (Discher, D. E., Mooney, D. J. & Zandstra, P. W., Science 324, 1673-1677, 2009 and Guilak, F. et al., Cell Stem Cell 5, 17-26, 2009), as well as in pathologies (Levental, K. R. et al., Cell 139, 891-906, 2009). These phenomena are mediated by focal adhesions and the associated coupling to microfilaments (Parsons, J. T., Horwitz, A. R. & Schwartz, M. A., Nat. Rev. 11, 633-643, 2010). Hence the control of the matrix mechanical properties is important for the design of biomaterials for stem cell expansion and for in vitro models and tissue engineering platforms.

EP0085573 (Keese & Giaever) reports that fibroblasts proliferate at relatively high rates on liquid substrates (using fluorocarbon liquid dispersed in aqueous polylysine solution in the absence of a surfactant. However, culture of other cell types, such as keratinocytes and MSC, rupture and destabilise the oil-aqueous interface after only a few days of culture. Papers by the same authors include Keese, C. R. & Giaever, I. Substrate mechanics and cell spreading. Exp. Cell Res. 195, 528-532 (1991) and Keese, C. R. & Giaever, I. Cell growth on liquid interfaces: Role of surface active compounds. Proc. Natl. Acad. Sci. 80, 5622-5626 (1983).

Kong et al., “The culture of HaCaT cells on liquid substrates is mediated by a mechanically strong liquid-liquid interface”, Faraday Discussions, published online 6 Apr. 2017 discusses the culture of HaCaT cells on liquid substrates. However, the methods and culture conditions are unsuitable for the culture of other cell types, such as primary keratinocytes, mesenchymal stem cells (MSCs), embryonic stem cells (ESCs) and induced pluripotent stem cells (iPSCs), for example because interface between the oil and liquid phase is not sufficiently adhesive to the cells or mechanically stable.

Although previous studies have shown that adherent cells (fibroblasts, HaCaT) can be expanded to high density at liquid-liquid interfaces, the cell culture systems of the art are not suitable for the long-term proliferation of a broader range of cell types. There remains a need in the art for a method and system for the successful culture of adherent cell types, including primary keratinocytes, MSCs, ESCs and iPSCs, on liquid-liquid interfaces to allow the proliferation and expansion of such cell types without the need for a solid substrate. Minami, K. et al. “Suppression of myogenic differentiation of mammalian cells caused by fluidity of a liquid-liquid interface”, Appl. Mater. Interfaces 9, 30553-30560 (2017) discusses culture of cells and liquid-liquid interfaces, but the systems do not involve the use of a surfactant, and the applicability of the system is limited. Hanga, M. P. et al. “Expansion of bone marrow-derived human mesenchymal stem/stromal cells (hMSCs) using a two-phase liquid/liquid system”, J. Chem. Technol. Biotechnol. 92, 1577-1589 (2017) discusses culture of cells at liquid-liquid interfaces, but again does not involve the use of a surfactant and results could not be reproduced by the present inventors (there was no growth of MSCs following the protocol described and in the absence of surfactant).

Here the present inventors show that integrin-mediated cell spreading, proliferation and control of fate decision occur at the surface of non-viscous liquids and are enabled by the self-assembly of mechanically strong nanoscale protein layers at these interfaces. These findings allow the reliable culture of adherent cells at liquid-liquid interfaces, providing cell culture systems with much increased surface area allowing a larger number of cells to be cultured, and allowing cell detachment without the need for enzymatic treatment. These findings also have important implications for the understanding of cellular mechanosensing, but also call for a shift in paradigm in the design of biomaterials used for regenerative medicine as they demonstrate that bulk and nanoscale mechanical properties may be designed independently to regulate cell adhesion and phenotype. This may find direct application for the development of 3D bioreactors and in cell sheet engineering.

### SUMMARY OF THE INVENTION

In a first aspect of the invention there is provided a method of culturing adherent cells at a liquid-liquid interface in a cell culture system, the cell culture system comprising:
a) an aqueous cell culture medium; and
b) an oil phase;
   wherein the oil phase is functionalised with a conditioning layer that is disposed between the aqueous cell culture medium and the oil phase and comprises a surfactant and a protein or peptide layer, the method comprising culturing the adherent cells in the cell culture system at the interface between the oil phase and the aqueous cell culture medium.

In a second aspect of the invention there is provided a cell culture system comprising
a) an aqueous cell culture medium; and
b) an oil phase;
   wherein the oil phase is functionalised with a conditioning layer that is disposed between the aqueous cell culture medium and the oil phase and comprises a surfactant and a protein or peptide layer. The cell culture systems are useful in the culture of adherent cells using the methods disclosed herein. The cell culture systems also comprise suitable serum, growth factors and any other chemicals required to promote cell growth, as on solid substrates.

In a third aspect of the invention there is provided the use of the cell culture system of the invention for the culture of adherent cells at a liquid-liquid interface.

In a fourth aspect of the invention there is provided a method of expanding a population of adherent cells comprising the culture of cells at a liquid-liquid interface according to the method of the invention and harvesting the cells from the culture medium.

In a fifth aspect of the invention, there is provided a population of cells cultured or expanded according to a method of the invention.

In a further aspect of the invention, there is provided a population of cells cultured or expanded according to a method of the invention for use in medicine.

In a further aspect of the invention there is provided a bioreactor comprising a culture of adherent cells, wherein the adherent cells are adhered to a liquid-liquid interface in a cell culture system of the invention.

In another aspect, there is provided a population of cells obtained by a method of the invention.

In another aspect of the invention there is provided a kit of parts comprising combinations of surfactants, oils, proteins and optionally polymer useful in the culture of adherent cells.

### BRIEF DESCRIPTION OF THE FIGURES

FIG. 1. Cell proliferation on low viscosity liquids is mediated by surfactants. A. HaCaT cells proliferation on a fluorinated oil (Novec 7500, 0.77 cSt) containing the surfactant pentafluorobenzoyl chloride at different concentrations (conditioning with medium). B. HaCaT cell proliferation profile on interfaces conditioned with BSA (1 mg/mL; blue diamonds, TPS; red square, Novec 7500+0.01 mg/mL PFBC; green triangles, Novec 7500+0.005 mg/mL PFBC). C. HaCaT cell proliferation on silicone oils with viscosities in the range of 10-5000 cSt and conditioned with BSA (1 mg/mL; blue (left bar in each pair), Day 3; red (right bar in each pair), Day 7; TPS, tissue culture polystyrene; ‘−’, no surfactant added; ‘+’, 0.5 mg/mL octanoyl chloride added; 5000/1000/50/10 describe the viscosity (cSt) of the PDMS oil used). Error bars are s.e.m.; n=3. Images are nuclear stainings (Hoechst, scale bars are 200 μm).

FIG. 2. Protein adsorption at the surface of fluorinated oils forms a strong nanoscale mechanical interface. A. Evolution of interfacial shear moduli and surfactant composition as a function of PFBC concentration. Blue diamonds and red squares are the interfacial shear moduli before and after the adsorption of BSA (1 mg/mL), respectively (error bars are s.e.m.; n=3). Green triangles are the surfactant compositions of protein assemblies determined from XPS (expressed as number of surfactant/BSA protein; error bars are experimental errors, 10%). B. SEM images of oil droplets ([S]=0.01 mg/mL, BSA=1 mg/mL), dried onto silicon substrates. C. Interfacial shear moduli of poly(L-lysine)-based interfaces generated at pH 7.4 and 10.5. Left bar in each pair is storage modulus and right bar in each pair is loss modulus. D and E. AFM characterisation (height image, profile and quantification of thickness) of oil droplets ([S]=0.01 mg/mL, BSA=1 mg/mL), dried onto silicon substrates. Error bars are s.e.m.; n>50. F. Schematic representation of protein deposition at oil interfaces. G. Structure of PFBC and the structure of PFBC covalently bound to proteins via amides. H. Schematic representation of a cell applying forces across an oil-water interface in the normal and tangential directions. I. Stiffness of fluorinated oil-water interfaces with varying BSA and surfactant (PFBC) concentrations measured by interfacial AFM with a colloidal probe (indentation depths were between 500-1000 nm at a frequency of 1 Hz; error bars are s.e.m; n=900).

FIG. 3. Cell spreading at liquid-liquid interfaces is mediated by integrin adhesion and regulated by acto-myosin contractility. A. Human primary keratinocyte (HPK) spreading (after 24 h) on TPS and PLL-fibronectin functionalised oil interfaces (functionalised with PLL at pH 7.4 or 10.5, then fibronectin at neutral pH). B. Corresponding fluorescence images (Red, actin; Blue, DAPI). C. HaCaT cells spreading on BSA interfaces (TPS and Novec 7500+PFBC) is modulated by the action of acto-myosin inhibitors (myosin inhibitor blebbistatin, 10 μM; ROCK inhibitor Y27632, 10 μM; actin polymerisation inhibitor Cytochalasin D, 1 μM). Cell areas determined from actin stainings (phalloidin). Left bar in each pair is TPS and right bar in each pair is oil+[S]. D. Blocking of β₁ integrins in HPK cells spreading on PLL-FN interfaces (TPS and Novec 7500+PFBC; blocking with mouse anti-(β₁ integrin antibody P5D2, 1:50, 20 μg/mL). PLL deposition was carried out in pH 10.5 PBS at the surface of fluorinated oil. Cell areas determined from actin stainings (phalloidin). Left bar in each pair is TPS and right bar in each pair is oil+[S]. E. Corresponding fluorescence microscopy images (blue, DAPI; red, phalloidin). F. Confocal microscopy images of HPKs spreading (after 24 h) on TPS and PLL-functionalised oil interfaces. Zooms correspond to the dotted boxes. G. SICM imaging in hopping mode of HPKs spreading (after 24 h) on TPS and PLL-functionalised oil interfaces. Zooms correspond to the dotted boxes. Error bars are s.e.m.; n=3.

FIG. 4. Stem cell culture at liquid-liquid interfaces and cell sheets formation. A. HPK differentiation on solid (TPS functionalised with fibronectin, Fn, or PLL-PEG) and liquid interfaces (Novec 7500+0.01 mg/mL PFBC; oil-Fn-pH7, PLL at pH 7.4 then Fn; oil-Fn-pH10, PLL at pH 10.5 then Fn; oil-PEG-pH10, PLL-PEG at pH 10.5) in differentiation medium (FAD) and basal medium (KSFM). Error bars are s.e.m.; n=3. B. Fluorescence microscopy images (red, actin; green, involucrin; blue, DAPI) corresponding to some of these conditions. C. Schematic representation of cell culture on emulsions and cell sheet formation. D. Micrographs of MSCs cultured on emulsions for 7 days in growth medium (top bright field; middle and bottom, epifluorescence images of Hoechst stainings). E. Left two columns, HaCaT cell sheets formed on glass or PLL-functionalised oil (top view of confocal stacks and single plane showing structures observed at the basal side of the sheet); red, actin; blue, nuclei; green, vinculin. Right two columns, HPK sheets formed on glass and PLL-functionalised oil in FAD without and with ROCK inhibitor Y27632 (10 μM, note the wrinkling of cell sheets); epifluorescence microscopy; red, actin; blue, DAPI.

FIG. 5. Impact of conditioning of the liquid-liquid interface on HaCaT cell proliferation and viability. A. HaCaT proliferation profile on interfaces conditioned with collagen (20 μg/mL; blue diamonds, TPS; red square, Novec 7500+0.01 mg/mL PFBC; green triangles, Novec 7500+0.005 mg/mL PFBC). B. HaCaT cell viability (live, green (left bar in each pair); dead, red (right bar in each pair)) when cultured on interfaces conditioned with collagen type I (C) or BSA (B). For oil+PFBC (surfactant, S), the corresponding concentrations of surfactant are stated (in mg/mL). Error bars are s.e.m.; n=3.

FIG. 6. Keratinocyte proliferation and gene expression at oil interfaces. A. HPKs proliferation on low viscosity (10 cSt) silicone oil functionalised with PLL-fibronectin (Fn) at PH 10.5 (red (uppermost data points, except for the last datum point), 10 cSt PDMS+0.05 mg/mL octanoyl chloride with PLL, 100 μg/mL, followed by Fn, 10 μg/mL; blue, TPS, tissue culture polystyrene). Error bars are s.e.m.; n=3. B. Corresponding nuclear stainings at Day 1 and Day 7 (Hoechst, scale bar is 200 μm). C. Relative gene expression of keratinocytes on PLL-Fn functionalised silicone oil (10 cSt PDMS with 0.05 mg/mL octanoyl chloride, PLL, 100 μg/mL and FN, 10 μg/mL) compared to PLL-Fn coated TPS after 7 day culture in KSFM. The reference is HPKs cultured on collagen coated TPS in FAD medium and GAPDH was used as internal reference. Relative gene expression was calculated via the 2^{-ΔΔCt} method.

FIG. 7. Human keratinocyte proliferating at the surface of oil droplets. Micrographs (confocal microscopy; top, actin in red; bottom, overlay of bright field, actin in red and nuclei in blue) of HPKs cultured on emulsions for 7 days in KSFM.

FIG. 8. Human MSCs proliferation and adhesion at oil interfaces. A. MSCs proliferation profile on fluorinated oil interfaces functionalised with PLL-Fibronectin (Fn) at pH 10.5 (PLL, 100 μg/mL; Fn, 10 μg/mL; red (lowermost data points), TPS; blue (uppermost data points), Novec 7500+0.00125 mg/mL PFBC). Error bars are s.e.m.; n=3. Images are corresponding nuclear stainings at Day 5 (Hoechst, scale bars are 200 μm). B. Confocal microscopy images of MSCs spreading (after 24 h) on TPS and PLL-functionalised oil. Zooms correspond to the dotted boxes.

FIG. 9. MSCs cultured on oil droplets can transfer to tissue culture plastic substrates. A. Bright field image of MSCs transferring from emulsions to TPS, day 2. B. Epifluorescence microscopy images of cells cultured on TPS first (7 days) and then reseeded on PLL-FN coated glass slides, after trypsin-induced detachment, and cells cultured on oil (7 days on Novec 7500+0.00125 mg/mL PFBC with PLL 100 μg/mL; Fn, 10 μg/mL) and reseeded on PLL-FN coated glass slides (red, actin; green, vinculin; blue, DAPI).

FIG. 10. Characterisation of cell sheets formed at liquid-liquid interfaces. A. Confocal microscopy images of HaCaT cell sheets formed on glass or PLL-functionalised oil. Left column, confocal stacks; middle and right columns, vinculin and actin images taken from a basal slice of the corresponding stack. The dotted boxes correspond to the zoom shown in FIG. 4E. B. Confocal microscopy images (right column, stacks; middle column, apical slie; right column, basal slice) of HPK sheets formed on glass and PLL-functionalised oil in FAD without and with ROCK inhibitor Y27632 (1 μM). Involucrin, green; nuclei, blue.

FIG. 11. Stress relaxation of fluorinated oil interfaces in the presence of 0.01 mg/mL of different surfactants (1H,1H,2H,2H-perfluoro-1-decanol, 1H,1H,2H-perfluoro-1-decene, 1H,1H-perfluorooctylamine, pentadecafluorooctanoyl chloride, perfluorodecanoic acid, octanoyl chloride, sebacoyl chloride and PFBC) after BSA deposition. Error bars are s.e.m.; n=3.

FIG. 12. HPK cells proliferation profile on fluorinated oil interfaces functionalised with PLL-fibronectin (Fn) at PH 10.5 (PLL, 100 μg/mL; Fn, 10 μg/mL; blue (uppermost data points, except for the first datum point), TPS; red, Novec 7500+0.01 mg/mL PFBC, TPS, tissue culture polystyrene). Error bars are s.e.m.; n=3. B. Images are nuclear stainings at Day 1 and Day 10 (Hoechst, scale bars are 200 μm).

FIG. 13. Stress relaxation of fluorinated oil interfaces after A. PLL deposition at PH 7.5 or PH 10.5; B. BSA deposition. [S], PFBC at a concentration of 0, 0.00125, 0.0025, and 0.01 mg/mL. Error bars are s.e.m.; n=3. Left bar in each pair is 1% strain, right bar in each pair is 0.05% strain.

FIG. 14. Cell proliferation on interfaces prepared with different surfactant concentrations. MSCs proliferation on PLL-FN coated fluorinated oil (Novec 7500, 0.77 cst) containing PFBC at different concentrations. Representative images of MSCs cultured on these interfaces for 5 days. Relative gene expression of MSCs on PLL-Fn coated fluorinated oil droplet containing PFBC at the concentration of 0.00125 μg/ml to on TPS after 7-day culture in growth medium, β-actin as the housekeeping gene and the relative gene expression was calculated by 2^{-ΔΔCt} method.

FIG. 15. A. HPK cells proliferation on silicone-based liquids functionalised with PLL-fibronectin (Fn) at PH 10.5 (PLL, 100 μg/mL; Fn, 10 μg/mL; blue (lowermost data points, except for the last datum point), TPS; red, 10 cSt PDMS+0.05 mg/mL octanoyl chloride; TPS, tissue culture polystyrene). Error bars are s.e.m.; n=3. B. Images are nuclear stainings at Day 1 and Day 7 (Hoechst, scale bars are 200 am). C. Relative gene expression of keratinocytes on PLL-Fn coated PDMS droplets; PLL-Fn coated TPS to on TPS after 7-day culture in KSFM. Keratinocytes cultured on PLL-PEG coated in TPS for 24 h in FAD was used as positive control. GPDH was used as housekeeping gene and the relative gene expression was calculated by 2^{-ΔΔCt} method.

FIG. 16. A. Confocal microscopy images of MSCs spreading (after 24 h) on TPS and PLL-functionalised oil interfaces. Zooms correspond to the dotted boxes. B. MSC cells proliferation profile on fluorinated oil interfaces functionalised with PLL-fibronectin (Fn) at PH 10.5 (PLL, 100 μg/mL; Fn, 10 μg/mL; red (lowermost data points), TPS; blue (uppermost data points), Novec 7500+0.00125 mg/mL PFBC, TPS, tissue culture polystyrene). Error bars are s.e.m.; n=3. B. Images are nuclear stainings at Day 5 (Hoechst, scale bars are 200 am).

FIG. 17. Cell transferred from emulsion to glass coated with PLL-Fn. A. Immunostaining images of MSCs grew on TPS-Fn and transferred from PLL-Fn coated oil; B. bright field imaging of cell transferred from the emulsions to PLL-Fn coated glass.

FIG. 18. Human primary keratinocyte cell proliferations on silicon-based liquids conditioned with different protein added with octanoyl chloride as surfactant. TPS, tissue culture polystyrene; oil, 10 cSt defined liquid PDMS; O, octanoyl chloride; PLL-FN, poly (L-lysine) adsorption (100 μg/mL) followed with fibronectin adsorption (10 μg/mL); PLL-PSS L-C, poly (L-lysine) adsorption (100 μg/mL) followed with Poly(sodium 4-styrenesulfonate) (MW: 70,000) adsorption (100 μg/mL) then followed with collagen type I adsorption (20 μg/mL). Error bars are s.e.m.; n=3.

FIG. 19. 0.05s indicates that the surfactant (octanoyl chloride) was used at a concentration of 0.05 mg/mL.

FIG. 20. Human mesenchymal stem cells (MSCs) proliferation on fluorinated oil interfaces containing 0.01 mg/mL PFBC deposited with different protein at Day 3 and Day 7. In all cases (apart from TPS control and Oil-Medium), the interfaces rely on the presence of PFBC at a concentration of 0.01 mg/mL.TPS, tissue culture polystyrene; oil, Novec 7500, 0.77 cSt; PLL-FN, poly (L-lysine) adsorption (100 μg/mL) followed with fibronectin adsorption (10 μg/mL); PLL-PSS(H)-Collagen, poly (L-lysine) adsorption (100 μg/mL) followed with poly(sodium 4-styrenesulfonate) (MW: 1000,000) adsorption (100 μg/mL) then followed with collagen type I adsorption (20 μg/mL); PLL-PSS(L)-Collagen, poly (L-lysine) adsorption (100 μg/mL) followed with Poly(sodium 4-styrenesulfonate) (MW: 70,000) adsorption (100 μg/mL) then followed with collagen type I adsorption (20 μg/mL); oil-medium, no protein functionalisation at the oil interfaces; PLL:BSA (1:1)-FN, poly (L-lysine) (100 μg/mL) and BSA (100 μg/mL) mixture adsorption followed with fibronectin adsorption (10 μg/mL); PLL:BSA (3:1)-FN, poly (L-lysine) (100 μg/mL) and BSA (33.3 μg/mL) mixture adsorption followed with fibronectin adsorption (10 μg/mL); ELP(+)-FN, ELP (+) (elastin like protein, positively charged, (VPGIG VPGIG VPGKG VPGIG VPGIG)24) adsorption (100 μg/mL) followed with fibronectin adsorption (10 μg/mL); PLL-ELP(−)-Collagen, poly (L-lysine) adsorption (100 μg/mL) followed with ELP(−) (elastin like kprotein, negatively charged, MESLLP-[(VPGVG VPGVG VPGEG VPGVGVPGVG)10-(VGIPG)60-V]) adsorption (100 μg/mL) then followed with collagen type I adsorption (20 μg/mL); ELP-RGD, elastin like protein, positively charged, containing RGD, [[(VPGIG)2(VPGKG)(VPGIG)2]2AVTGRGDSPASS[(VPGIG)2(VPGKG)(VPGIG)2]2]6, 100 μg/mL; PLL-HA(H)-Collagen, poly (L-lysine) adsorption (100 μg/mL) followed with Hyaluronic acid (MW: 700,000) adsorption (100 μg/mL) then followed with collagen type I adsorption (20 μg/mL); PLL-HA(L)-Collagen, poly (L-lysine) adsorption (100 μg/mL) followed with Hyaluronic acid (MW: 60,000) adsorption (100 μg/mL) then followed with collagen type I adsorption (20 μg/mL). Error bars are s.e.m.; n=3. Left bar in each pair is day 3, right bar in each pair is day 7.

FIG. 21. Representative images of cultures on: A. TPS (Tissue culture polystyrene); B. PLL-FN; C. PLL-PSS-Collagen (where PSS is poly(styrene sulfonate); D. 4. PLL-HA-Collagen (where HA is hyaluronic acid). Although confluency is not reached as fast, the data demonstrate the proliferation of MSCs on fluorinated oils.

FIG. 22. Epifluorescence images of labelled oil interfaces and cells. Human primary keratinocytes were seeded at the labelled oil interfaces (labelled PLL under PH 10.5) in KSFM medium. red, actin; blue, nuclear.

FIG. 23. MSCs cultured on emulsions (fluorinated oils) functionalised with PFBC and PLL-fibronectin.

FIG. 24. Stress relaxation of fluorinated oil Novec 7500 in the presence of 0.01 mg/mL PFBC after BSA (1 mg/mL) adsorption at 1% strain (strain rates from 0.05 to 0.1%/s). The data was fit from the onset of relaxation, using a double exponential model (Eq1). The level of elasticity was determined from the ultimate stress remaining within the interface at infinite time point (Eq2).

FIG. 25. Proliferation profile of MSCs grown on Novec 7500 with different PFBC concentrations on 3-day culture. The Novec 7500 interface was prepared with different PFBC concentrations (0.01 mg/ml, 0.005 mg/ml, 0.0025 mg/ml and 0.00125 mg/ml) and treated with PLL and fibronectin. Cells were also cultured on TPS as control.

FIG. 26. Proliferation profile of MSCs grown on FC-40 with different PFBC concentrations on 3-day culture. The FC-40 interface was prepared with different PFBC concentrations (0.01 mg/ml, 0.005 mg/ml, 0.0025 mg/ml and 0.00125 mg/ml) and treated with PLL and fibronectin. Cells were also cultured on TPS as control

FIG. 27. Proliferation profile of MSCs grown on Novec 7500 with different pentadecafluorooctanoyl chloride concentrations on 3-day (blue (left bar in each pair)) and 7-day (brown (right bar in each pair)) culture. The Novec7500 interface was prepared with different pentadecafluorooctanoyl chloride concentrations (0.01 mg/ml, 0.005 mg/ml, 0.0025 mg/ml and 0.00125 mg/ml) and treated with PLL and fibronectin. Cells were also cultured on TPS as control.

FIG. 28. Hoechst staining of MSCs grown on PDMS droplet on day 10. The droplets were prepared with 0.1 mg/ml Heptadecanoyl chloride (A. 2.5× and B. 10×) and 0.1 mg/ml Heptadecanoyl chloride and Sebacoyl chloride (3:1) mixture (C. 2.5× and B. 10×) treated with PLL and fibronectin

FIG. 29. HPKs proliferation on fluorinated oil (Novec 7500, 0.77 cSt) interfaces (with PFBC at different concentrations). Interface containing the surfactant PFBC at different concentration deposited with PLL-FN, PLL adsorption (100 μg/mL) followed with fibronectin adsorption (10 μg/mL); Error bars are s.e.m.; n=3. Images are nuclear stainings (Hoechst, scale bars are 200 μm) at Day 7.

FIG. 30. MSCs proliferate on silicone oil. MSCs proliferation on PLL-FN coated PDMS oil (10 cSt) containing 0.1 mg/mL sebacoyl/heptadecanoyl chloride mix at 3:1 ratio. Representative images of MSCs cultured on these interfaces at different time points. MSCs proliferation profile on these interfaces.

FIG. 31. HPKs proliferation on silicone oil conditioned with different nanosheets (multilayers functionalised with fibronectin or collagen) generated with 0.05 mg/mL octanoyl chloride as surfactant. PLL is poly(L-lysine), PSS is poly(styrene sulfonate), FN is fibronectin, C is collagen. Images are nuclear staining (Hoechst, scale bars are 200 μm) at Day 7. Bar chart is corresponding to cell density of the images. Error bars are s.e.m.; n=3. n.s. indicates no significant difference between the different interfaces at each time point.

FIG. 32. MSCs proliferate on silicone oil with different surfactant mixtures. MSCs proliferation on PLL-FN coated PDMS oil (10 cSt) containing 0.01 mg/ml sebacoyl (S)/heptadecanoyl chloride (H) mixtures at different ratios. 1:1 and 3:1 refer to the corresponding S:H ratios. Representative images of MSCs cultured on these interfaces for 3 days and 5 days. MSCs proliferation profile on these interfaces, Error bars are s.e.m.; n=4.

FIG. 33. MSCs proliferate on rapeseed oil functionalised with 0.01 mg/ml sebacoyl/heptadecanoyl chloride mixture at 1:1 ratio. MSCs proliferation on PLL-FN coated rapeseed oil containing 0.01 mg/ml sebacoyl/heptadecanoyl chloride mix at 1:1 ratios. Representative images of MSCs cultured on these interfaces for 3 days and 7 days. MSCs proliferation profile on these interfaces, Error bars are s.e.m.; n>3.

FIG. 34. MSCs proliferate on mineral oil with 0.01 mg/ml sebacoyl/heptadecanoyl chloride mix at 1:1 ratio. MSCs proliferation on PLL-FN coated mineral oil containing 0.01 mg/mL sebacoyl/heptadecanoyl chloride mixture at 1:1 ratios. Representative images of MSCs cultured on these interfaces for 3 days and 5 days. MSCs proliferation profile on these interfaces, Error bars are s.e.m.; n>3.

FIG. 35. MSCs proliferate on rapeseed oil with 0.1 mg/ml heptadecanoyl chloride. MSCs proliferation on PLL-FN coated rapeseed oil containing 0.1 mg/mL heptadecanoyl chloride. Representative images of calcein staining MSCs cultured on these interfaces for 3 days and 7 days, confirming the high viability of cells at these interfaces.

FIG. 36. MSCs cultured on silicone oil droplets. 1 mL PDMS (10 cSt) containing sebacoyl/heptadecanoyl chloride mix at 1:1 ratio at 0.01 mg/ml concentration and 2 mL of PLL solution (200 μg/mL) in pH10.5 PBS were added in a glass vial. The vial was vigorously shaken to mix and form the emulsion and subsequently left to incubate at room temperature for 1 h. The bottom liquid phase below the settled emulsion was aspirated and replaced with PBS 4 times. 20 μL of human plasma fibronectin (1 mg/mL) was added (final concentration of 10 μg/mL) and incubated at room temperature for 1 h. The bottom liquid phase below the emulsion was aspirated and replace with PBS 3 times. For cell seeding, 2 mL of growth medium was added in a 24 well plate and 500 μL of the emulsion were transferred to the well. Epifluorescence images of calcein staining of MSCs cultured on emulsion for 7 days in growth medium.

FIG. 37. HPK cell adhering (when seeded at high density, 300 k/well, after 24 h) at Fluorinated oil (Novec 7500, 0.77 cSt) interface deposited with (A and B) and without PLL-GO (graphene oxide) composites (C). Interface containing the surfactant PFBC 0.00125 mg/mL. PLL-GO composites, PLL adsorption (100 μg/mL) followed with graphene oxide adsorption (100 μg/mL) 3 times then followed with PLL adsorption (100 μg/mL) and fibronectin adsorption (10 μg/mL); PLL only, PLL adsorption (100 μg/mL) followed with fibronectin adsorption (10 μg/mL). Images are fluorescence images (Red, tagged PLL; Blue, nuclei).

FIG. 38. The culture of MSCs at the surface of two fluorinated oils (FC-40 and Novec 7500), in the absence of protein nanosheets deposition, but with conditioning with cell culture medium (following the protocol reported by Hanga et al. 2017²⁷), did not lead to any significant cell proliferation after 7 days of culture).

FIG. 39. iPSCs proliferate on fluorinated oil. iPSC proliferation on PLL-vitronectin coated Novec 7500 oil containing 0.00125 mg/mL PFBC. Top, representative images of iPSCs cultured on these interfaces at different time points. Bottom, representative images of colonies via immunofluorescence (F-actin, nuclei, calcein), at day 5.

FIG. 40. Images of microdroplets stabilised by BSA nanosheets assembled using a microdroplet microfluidic system, with Novec 7500 oil and albumin (1 mg/mL), in the presence of 0.01 mg/mL PFBC. Fluorescence images of microdroplets generated using Novec 7500 oil and PLL solutions (100 μg/mL), at pH 10.5, using PFBC (0.01 mg/mL). PLL nanosheets were tagged with Alexa-fluor 594-functionalised PLL (10%) and images of MSCs cultured at the surface of the resulting emulsions (two images on the right bottom, calcein staining after 7 days culture)

FIG. 41. Oscillatory rheology characterisation of mineral oil—PBS interfaces by a) Oscillating frequency sweep with a oscillating displacement of 10-4 rad from 0.01-10 Hz on mineral oil—PBS interfaces with and without lysozyme (used at a concentration of 10 mg/mL) and benzoyl chloride (used at a concentration of 0.1 mg/mL) respectively, where the solid dots are G′ and hollow dots are G″, b) stress relaxation data on interfaces showing the impact of protein on the relaxation profile, c) representative time sweep showing the formation of a lysozyme protein film on a mineral oil—PBS interface (solid line showing G′ and the dotted line G″), d) summary of the interfacial mechanics of mineral oil—PBS interfaces comparing films with and without surfactant and lysozyme respectively. A minimum of three samples was tested per test. All error bars are standard deviations.

FIG. 42. The cell culture systems may comprise or consist of any of the arrangement of components depicted (beginning in each case with the component or layer adjacent to the oil phase, i.e. the first layer). A) the conditioning layer comprises a surfactant (e.g. a non-polymeric surfactant) with a protein/peptide layer. B) the conditioning layer comprises surfactant that is a polymer and a separate, different, protein/peptide layer. C) the conditioning layer comprises a surfactant (e.g. a non-polymeric surfactant) with at least one polymer layer (optionally a non-peptidic polymer), and a separate, different, protein/peptide layer. D) the conditioning layer comprises a surfactant that is a polymer, at least one additional polymer layer (optionally a non-peptidic polymer), and a protein/peptide layer, wherein the surfactant, the polymer of the additional polymer layer, and the protein/peptide layer, are all different. E) the conditioning layer comprises a surfactant (e.g. a non-polymeric surfactant) with a first polymer layer (optionally a non-peptidic polymer), a second, different, polymer layer (optionally a non-peptidic polymer), and a protein/peptide layer. F) the conditioning layer comprises a polymeric surfactant, at least two additional polymers (optionally a non-peptidic polymer), and a protein/peptide layer, wherein the surfactant, each of the at least two additional polymers and the protein/peptide layer are different.

### DETAILED DESCRIPTION

### Cell Culture Systems

The methods of the present invention employ a novel cell culture system that provides optimal conditions for the culture of adherent cells at a liquid-liquid interface. The cell culture systems are particularly suited to the culture of adherent stem cells, although any adherent cell types can be used. Cell populations cultured according to methods of the invention grow just as well, and in some cases better, compared to traditional cell culture systems that use a solid substrate such as plastic.

The cell culture systems comprise an aqueous cell culture medium and an oil phase. The oil phase comprises a conditioning layer that is assembled between the aqueous cell culture medium and the oil phase. The conditioning layer comprises a protein or peptide layer and a surfactant. The conditioning layer functionalizes the oil phase to allow the efficient and longer-term culture of adherent cells. When cells are cultured, they grow on the surface of the functionalised oil (i.e. on the conditioning layer). Unlike in cell cultures systems of the prior art, the cell culture systems of the invention allow the culture of the cells at the liquid-liquid interface without causing disruption of the surface of the oil by providing optimum stress-relaxation conditions, since the inventors have surprisingly found the energy can be stored in the form of elastic energy (due to the lower modulus of the interface) rather than being dissipated through fracture. Contrary to the wisdom in the art, the inventors found that a high modulus is not required for the culture of the cells at the liquid-liquid interface, and indeed would be detrimental to the culture of cells given such a higher modulus makes the interface brittle. The cells may adhere via integrin-mediated adhesion and cytoskeleton assembly.

The cell culture systems of the present invention all comprise a surfactant, a polymer, and a protein forming a conditioning layer. However, given the various possible alternative components, the surfactant itself can be the polymer, or the protein can be the polymer. The minimum requirements of the conditioning layer are a surfactant and a polymer layer, wherein the surfactant is bonded to the polymer layer by covalent and/or supramolecular forces. If the polymer layer is a protein, this can act as the protein layer. If the polymer layer is a non-peptidic polymer layer, then an additional protein layer is required. The protein layer is adhesive to adherent cells. The conditioning layer is provided such that it has the suitable mechanical properties discussed herein to enable to long-term culture of adherent cells, in particular stem cells.

The present culture systems and methods allow the culture of adherent cells in a scalable system that can easily be used to provide a large number of cultured cells due to the increased proliferation rate of cells cultured using the system, and the increased surface area of systems that are in the form of an emulsion. The cell systems presented may also allow the production of a large amount of proteins or other molecules synthesised by the cells, for example the production of antibodies or recombinant proteins and growth factors by cells. The cell culture systems also allow the longer-term culture of cells such as MSCs and HPKs than seen with the cell culture systems of the art.

### Oil Phase

The choice of oil will depend on a number of factors, such as the surfactant used and the type of cells that are to be cultured. Generally, the oil will be an oil selected from the group consisting of a silicone oil, a fluorinated oil, a hydrocarbon, a paraffin oil, a mineral oil, a fatty acid oil, castor oil, palm oil, rapeseed oil and olive oil. In some embodiments, the oil will be an oil selected from the group consisting of a silicone oil, a fluorinated oil, a hydrocarbon, a paraffin oil, a fatty acid oil, castor oil, palm oil and olive oil. Of particular relevance to the present invention are silicone oils and fluorinated oils.

In one embodiment of the invention, the oil is selected from a silicone oil and a fluorinated oil. The successful use of silicone oils, for example when used in combination with PFBC and other non-fluorinated acyl chlorides, is particularly surprising since cell culture systems of the prior art where not able to establish proliferation of cells such as stem cells using silicone oils (e.g. Keese & Giaever, Science, 219:1448-1449, 1983 and Keese & Giaever, Proc. Natl. Acad. Sci., 80:5622-5626, 1983). In one embodiment, the silicone oil is polydimethylsiloxane (PDMS) or an associated derivative thereof (for example, vinylated, thiolated, alkylated and aromatic substituted derivatives thereof). A broad range of molecular weights and viscosities of PDMS can be used in the present invention. For example, in one embodiment, the oil is PDMS having a viscosity from 5 cSt to 5000 cSt.

In one embodiment, the oil may be rapeseed oil. Rapeseed oil includes oils such as canola oil or colza oil.

In one embodiment, the oil may be a mineral oil. A mineral oil may be a colourless, odourless, light mixture of higher alkanes from a mineral source, for example a distillate of petroleum. Mineral oils include, but are not limited to, oils known as liquid petroleum, paraffinum liquidum, liquid paraffin paraffin oil and white oil. The mineral oil may be hexadecane.

In embodiments where a fluorinated oil is used, the oil may be Novec 7500 or FC-40.

Novec 7500 is hexane, 3-ethoxy-1,1,1,2,3,4,4,5,5,6,6,6-dodecafluoro-2-(trifluoromethyl) (also known as 2-(Trifluoromethyl)-3-ethoxydodecafluorohexane, CAS No. 297730-93-9):

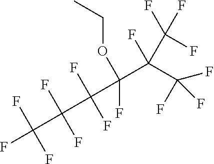 [SMILES: CCOC(F)(C(F)(F)C(F)(F)C(F)(F)F)C(F)(C(F)(F)F)C(F)(F)F]

FC-40 (also known as Fluorinert™ FC-40) is a mixture of 1,1,2,2,3,3,4,4,4-nonafluoro-N,N-bis(1,1,2,2,3,3,4,4,4-nonafluorobutyl)butan-1-amine and 1,1,2,2,3,3,4,4,4-nonafluoro-N-(1,1,2,2,3,3,4,4,4-nonafluorobutyl)-N-(trifluoromethyl)butan-1-amine, CAS No. 51142-49-5):

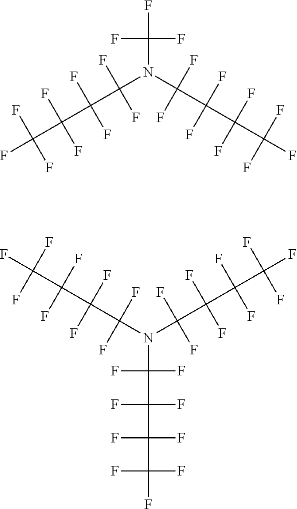 [SMILES: FC(F)(F)N(C(F)(F)C(F)(F)C(F)(F)C(F)(F)F)C(F)(F)C(F)(F)C(F)(F)C(F)(F)F ; FC(F)(F)C(F)(F)C(F)(F)C(F)(F)N(C(F)(F)C(F)(F)C(F)(F)C(F)(F)F)C(F)(F)C(F)(F)C(F)(F)C(F)(F)F]

The oil and the surfactant can be miscible to enable the oil to be suitably functionalised to allow the cultured cells to adhere to the surface of the oil phase. For example, the surfactant may have a solubility in the oil of at least 0.0001 mg/ml.

In some embodiments, the cell culture system is an emulsion (in particular, an oil-in-water emulsion). In such embodiments, the oil is present as a plurality of droplets contained within the aqueous cell culture media. The droplets may be microdroplets. Such embodiments enable a large number of cells to be cultured by providing a high surface area on which the cells can be cultured. The droplets may be from about 0.1 to about 500 μm in diameter.

In other embodiments, the oil phase and the aqueous media are not an emulsion, and instead the cell culture occurs as a planar sheet at the interface of the oil and aqueous phases. Such embodiments are useful when sheets of cells are desired for a given intended use. The planar sheet may have a surface area of at least 10 cm².

### Surfactant and Optional Additional Polymer Layers

Like the oil, the choice of surfactant will depend on a number of factors, including the oil used and the choice of other components of the cell culture system, in particular the conditioning layer.

The surfactant mediates strong interactions between the oil phase and the first layer (or only layer, if there is a single layer) of the conditioning layer. The strong interactions may be covalent or supramolecular bonds between the surfactant and the first layer of the conditioning layer. The “first layer” of the conditioning layer is the layer in direct contact with the oil phase. In cell culture systems having only one layer, the first layer is also in direct contact with the aqueous phase.

Covalent and/or supramolecular interactions may be achieved by the presence of one or more reactive groups. A “reactive group” is one that allows the formation of covalent or supramolecular bonds between the surfactant and the first layer of the conditioning layer. Therefore, the surfactant may comprise one or more reactive groups that are capable of forming covalent and/or supramolecular bonds between the surfactant and the first layer of the conditioning layer.

The reactive group of the surfactant is a reactive group that allows the formation of covalent or supramolecular bonds between the surfactant and the first layer in the conditioning layer. Thus, the precise choice of surfactant (and reactive group) may depend on the nature of the other components. Importantly, the components of the cell culture system should be chosen to allow the formation of covalent or supramolecular bonds between the surfactant and the relevant component or components of the first layer of the conditioning layer.

Functional groups that allow the formation of covalent bonds may be selected from the group consisting of activated carboxylic acids, activated carbonates, azides (for example for alkyne-azide click reactions), alkenes, alkynes, alkoxysilanes, ketoximes, acetoxysilanes. In addition, functional groups that can form supramolecular bonds with the polymers deposited at the interface may be selected from the group consisting of biotin, streptavidin, cyclodextrin, cucurbituril, cyclobis(paraquat-p-phenylene), short sequences of nucleic acid molecules (for example DNA, RNA or peptide-nucleic acid (PNA) molecules), such as sequences of nucleic acid molecules 1 to 10 residues in length, self-aggregating or self-assembling peptides, and peptides enabling specific binding to other molecules (such as antibodies).

Activated carboxylic acids refer to acids that allow coupling of the acid group to alcohols and amines, forming ester and amides. Appropriate activated carboxylic acids include, for example, acids activated with N-hydroxysuccinimide esters (NHS-esters), carbodiimides, hydroxybenzotriazole (HOBT), 1-[Bis(dimethylamino)methylene]-1H-1,2,3-triazolo[4,5-b]pyridinium 3-oxid hexafluorophosphate (HATU), or 4-(4,6-dimethoxy-1,3,5-triazin-2-yl)-4-methyl-morpholinium chloride (DMTMM)

Activated carbonates include, for example, carbonates activated with nitrophenyl chloroformate (NPC) or disuccinimidyl carbonate (DSC).

Supramolecular bonds include hydrogen bonding, electrostatic interactions and pi-pi stacking.

The conditioning layer may further comprise one or more polymers. The polymers of the conditioning layer may be non-peptidic polymers. Alternatively, if the polymer is a protein polymer, it may function as the protein/peptide layer.

In some embodiments, the surfactant is a polymeric surfactant. In particular, the surfactant may act as the polymer of the conditioning layer. In other embodiments, the polymer of the conditioning layer (if present) is a different polymer to the polymeric surfactant. In such embodiments, there are at least two polymer layers in the conditioning layer.

The protein/peptide layer, surfactant and optional polymer(s) can be arranged in a number of ways. Generally, the arrangement will be the oil, then one or more optional layers of one or more polymers, then the protein/polymer layer. The surfactant is situated at the first layer of the conditioning layer (the layer closest to the oil). If the surfactant is a polymeric surfactant, the surfactant represents the first polymer layer of the conditioning layer and the polymeric surfactant is bonded to the next polymeric layer via supramolecular and/or covalent bonds. If the surfactant is a non-polymeric surfactant, the surfactant is bonded to the first polymeric layer via supramolecular and/or covalent bonds.

In cases where a polymeric surfactant is used, the conditioning layer comprises at least two polymeric layers. The additional polymeric layers are a protein/peptide layer with optional additional polymeric layers situated between the polymeric surfactant and the protein/peptide layer. In cases where a non-polymeric surfactant is used, the conditioning layer comprises one or more polymeric layers. For a single-layer conditioning layer, the single layer is a peptide/protein layer. For conditioning layers having multiple layers, the additional layers are provided by the additional polymeric layers situated below the peptide/protein layer.

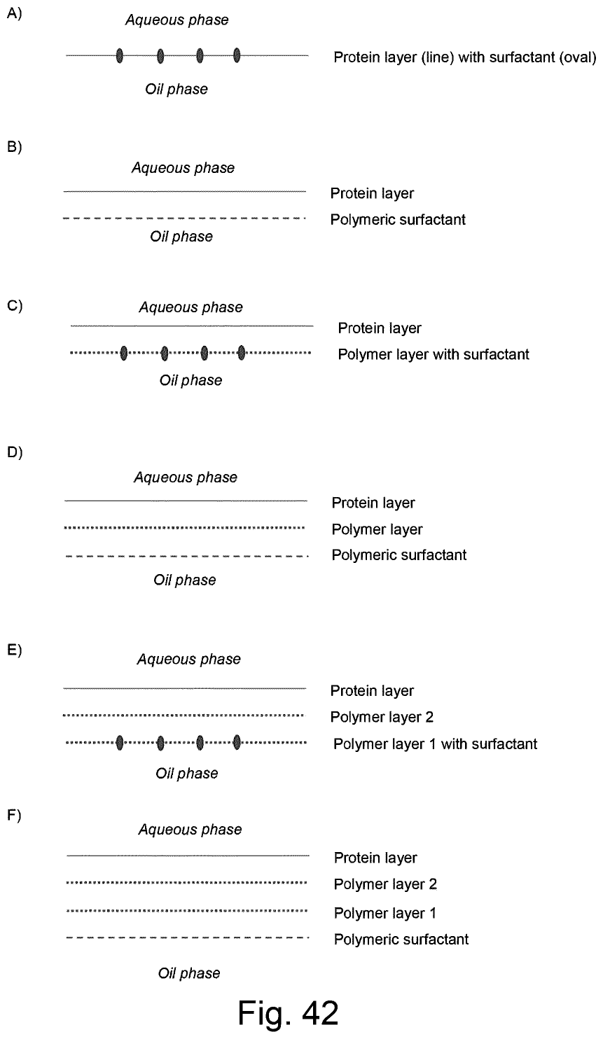

For example, the cell culture systems may comprise or consist of any of the arrangement of components depicted in FIG. 42A to 42F (beginning in each case with the component or layer adjacent to the oil phase, i.e. the first layer):
a) the conditioning layer comprises a surfactant (e.g. a non-polymeric surfactant) with a protein/peptide layer. It can be seen in FIG. 42A, in one such embodiment, the conditioning layer comprises a protein layer which itself comprises a non-polymeric surfactant adjacent to the oil phase. The components of the conditioning layer (protein layer and surfactant) are different from one another.
b) the conditioning layer comprises surfactant that is a polymer and a separate, different, protein/peptide layer. It can be seen in FIG. 42B, in one such embodiment, the conditioning layer comprises a protein layer adjacent to the aqueous phase, and a polymeric surfactant adjacent to the oil phase. The components of the conditioning layer (protein layer and surfactant) are different from one another.
c) the conditioning layer comprises a surfactant (e.g. a non-polymeric surfactant) with at least one polymer layer (optionally a non-peptidic polymer), and a separate, different, protein/peptide layer. It can be seen in FIG. 42C, in one such embodiment, the conditioning layer comprises a polymer layer disposed between the oil phase and the protein layer, and further wherein the polymer lay comprises the non-polymeric surfactant. Each of the components of the conditioning layer (protein layer, polymer layer, and surfactant) are different.
d) the conditioning layer comprises a surfactant that is a polymer, at least one additional polymer layer (optionally a non-peptidic polymer), and a protein/peptide layer, wherein the surfactant, the polymer of the additional polymer layer, and the protein/peptide layer, are all different. It can be seen in FIG. 42D, in one such embodiment, the conditioning layer comprises a polymer layer disposed between a polymeric surfactant and the protein layer. Each of the components of the conditioning layer (protein layer, polymer layer, and surfactant) are different.
e) the conditioning layer comprises a surfactant (e.g. a non-polymeric surfactant) with a first polymer layer (optionally a non-peptidic polymer), a second, different, polymer layer (optionally a non-peptidic polymer), and a protein/peptide layer. It can be seen in FIG. 42E, in one such embodiment, the conditioning layer comprises a first polymer layer disposed between the oil phase and a second, different, polymer layer. The second polymer layer is disposed between the first polymer layer and the protein layer. The first polymer layer comprises a non-polymeric surfactant. Each of the components of the conditioning layer (protein layer, first polymer layer, second polymer layer, and surfactant) are different. Additional polymer layers are possible by alternating layers for the first and second polymer between the oil phase and the protein layer.
f) the conditioning layer comprises a polymeric surfactant, at least two additional polymers (optionally a non-peptidic polymer), and a protein/peptide layer, wherein the surfactant, each of the at least two additional polymers and the protein/peptide layer are different. It can be seen in FIG. 42F, in one such embodiment, the conditioning layer comprises first and second polymer layers disposed between a polymeric surfactant and the protein layer. Each of the components of the conditioning layer (protein layer, first polymer layer, second polymer layer, and surfactant) are different. Additional polymer layers are possible by alternating layers for the first and second polymer between the polymeric surfactant and the protein layer.

Additional layers of polymers are possible. In each case, the surfactant, if it is non-polymeric (scenarios (a), (c) and (e) above), is bonded to the first layer of the conditioning layer via covalent and/or supramolecular forces. If the surfactant is polymeric (scenarios (b), (d) and (f) above), it is bonding to its adjacent layer (i.e. the second layer of the conditioning layer) via covalent and/or supramolecular forces. Covalent and/or supramolecular forces can be confirmed by, for example, XPS analysis and/or Fourier transform infrared spectroscopy (FTIR).

When additional layers beyond the simple protein/peptide layer are present in the conditioning layer, the protein layer is the outermost or top layer of the conditioning layer and is therefore disposed at the interface with the aqueous medium. Conversely, the surfactant is at the innermost or bottom layer of the conditioning layer and is therefore disposed at the interface with the oil phase.

In embodiments where the conditioning layer comprises at least two different polymers that are not acting as the surfactant (for example, as in scenarios (e) and (f) above), the polymers can be placed in an alternating arrangement to allow multiple layers of polymers to be incorporated into the conditioning layer. For example, the following arrangements are possible examples for scenario (e) above, starting from the layer in contact with the oil phase:
i. non-polymeric surfactant with a layer of a first polymer; layer of a second polymer; protein/peptide layer; (3 layers in total)
ii. non-polymeric surfactant with a layer of a first polymer; layer of a second polymer; additional layer of the first polymer; protein/peptide layer; (4 layers in total)
iii. non-polymeric surfactant with a layer of a first polymer; layer of a second polymer; additional layer of the first polymer; additional layer of the second polymer; protein/peptide layer; (5 layers in total)
iv. non-polymeric surfactant with a layer of a first polymer; layer of a second polymer; additional layer of the first polymer; additional layer of the second polymer; additional layer of the first polymer; protein/peptide layer; (6 layers in total)
   and so on. Similar arrangements are possible with scenario (f) above, using a polymeric surfactant instead of a non-polymeric surfactant.

In this way, the skilled person can provide a multi-layered conditioning layer. The protein/peptide layer supports the cells and so is present at the interface with the aqueous medium.

Accordingly, the cell culture system of the invention comprises a conditioning layer comprising a surfactant, optionally one or more layers of one or more polymers or additional polymers, and a peptide/protein layer. In a preferred embodiment, the cell culture system of the invention comprises a comprising a conditioning layer, the conditioning layer comprising a surfactant bonded to a protein/peptide layer, wherein the surfactant is bonded to the protein/peptide layer via covalent and/or supramolecular forces. In a more preferred embodiment, the conditioning layer comprises a non-polymeric surfactant, a polymer layer, and a separate, different, peptide/protein layer, wherein the surfactant is bonded to the polymer layer via covalent and/or supramolecular forces.

In one embodiment, there is provided a cell culture system having a multi-layered conditioning layer, the conditioning layer comprising a surfactant, at least two layers of alternating polymers (for example 3 or 4 layers of two alternating polymers), and a peptide/protein layer, wherein the surfactant is bonded to the first polymer layer via covalent and/or supramolecular forces.

There are many suitable surfactants that can be used. In some embodiments, the surfactant is an acyl chloride surfactant (for example pentafluorobenzoyl chloride, pentadecafluorooctanoyl chloride, octanoyl chloride, sebacoyl chloride or heptadecanoyl chloride).

In some embodiments, mixtures of surfactants can be used.

In embodiments where the cell culture system is an emulsion, the surfactant acts as an emulsifier.

The amount of surfactant can be measured as a concentration. Generally, the surfactant will be present in an amount of less than or equal to about 0.05 mg/ml or less than or equal to about 0.01 mg/ml. In some embodiments, the concentration of the surfactant is from about 0.001 mg/ml to about 0.05 mg/ml, or from about 0.00125 mg/ml and about 0.01 mg/ml. The concentrations are measured with respect to the total volume of oil used in the cell culture system (e.g. up to 0.001 mg of surfactant per 1 ml of oil).

The precise amount of surfactant may depend on the cells to be cultured and the other components of the system. For example, when using a combination of Novec 7500, PFBC, PLL and fibronectin (for example to culture MSCs), a PFBC surfactant concentration of from about 0.005 mg/ml to about 0.001 mg/ml may be preferred. When using a combination of FC-40, PFBC, PLL and fibronectin (for example to culture MSCs), a PFBC surfactant concentration of from about 0.01 mg/ml to about 0.001 mg/ml may be preferred. When using a combination of FC-40, pentadecafluorooctanoyl chloride, PLL and fibronectin (for example to culture MSCs), a pentadecafluorooctanoyl chloride surfactant concentration of from about 0.01 mg/ml to about 0.002 mg/ml may be preferred. The precise amounts can be adjusted by the skilled person according to the combination of components and the cell types being used.

Possible suitable combinations of oils and surfactants are as follows (this list is non-exhaustive):
the surfactant is pentafluorobenzoyl chloride and the oil is a fluorinated oil, such as 3-ethoxy-1,1,1,2,3,4,4,5,5,6,6,6-dodecafluoro-2-(trifluoromethyl).
the surfactant is octanoyl chloride and the oil is a silicone oil, such as polydimethylsiloxane.
the surfactant is sebacoyl chloride and the oil is a silicone oil, such as polydimethylsiloxane.
the surfactant is heptadecanoyl chloride and the oil is a silicone oil, such as polydimethylsiloxane.
the surfactant is heptadecanoyl chloride and the oil is rapeseed oil
the surfactant is a mixture of sebacoyl chloride and heptadecanoyl chloride and the oil is a silicone oil, such as polydimethylsiloxane.
the surfactant is a mixture of sebacoyl chloride and heptadecanoyl chloride and the oil is rapeseed oil
the surfactant is a mixture of sebacoyl chloride and heptadecanoyl chloride and the oil is mineral oil

Octanoyl chloride, sebacoyl chloride and heptadecanoyl chloride may be particularly useful in combination with silicone oils, and even more preferably is the combination of any of these three surfactants, a silicone oil, and the polymer PLL. Such a cell culture system is particularly useful for the culture of human keratinocytes. Alternatively, pentafluorobenzoyl chloride (PFBC) and/or pentadecafluorooctanoyl chloride may be useful in combination with a fluorinated oil and PLL. Surprisingly, such a cell culture system results in a decrease in interfacial mechanics at lower PFBC concentrations (for example up to 0.0025 mg/mL of oil or up to 0.00125 mg/mL of oil), yet is still suitable for the culture of stem cells such as MSCs due to an increase in elasticity. This correlates with the inventors' findings that it is not stiffness that is key to the culture of adherent cells, but rather elasticity.

Octanoyl chloride, sebacoyl chloride and heptadecanoyl chloride may also be useful in combination with oils such as rapeseed oils and mineral oils. Even more preferable is the combination of any of octanoyl chloride, sebacoyl chloride and heptadecanoyl chloride, a rapeseed or mineral oil, and the polymer PLL.

A non-fluorinated surfactant may be preferred when using non-fluorinated oils, contrary to previous cell culture systems of the prior art (studies by Keese and Giaever).

In some embodiments, the polymer is positively charged. A positively charged polymer may be beneficial to promote adsorption of extracellular matrix proteins produced by the culture cells, such as fibronectin or vitronectin. Example positively charged polymers include poly(lysine), poly(allyl amine), poly(ethylene imine) (linear or branched), chitosan and copolymers containing these motifs. However, other positively charged polymers could also be used.

The additional polymer layer, if present, may comprise a reactive group. When present, the reactive group in the polymer layer allows the formation of covalent and/or supramolecular bonds between the polymer and the surfactant. The reactive group in the polymer layer may be selected from the group consisting of an amine, an alcohol and a thiol group.

Both natural and synthetic polymers can be used. Example polymers that can optionally be included in the conditioning layer are poly(L-lysine) (PLL), poly(allylamine), poly(vinyl alcohol) (PVA), poly(hydroxyethyl methacrylate) (PHEMA), chitosan, poly(serine), dextran, heparin, poly(styrene sulfonate), chondroitin sulfate, hyaluronic acid, carboxy methyl cellulose, albumin, lysozyme, lactoglobulin, fibronectin, collagen, laminin, agrin, fibroin, elastin, elastin like proteins (ELPs), resilin, sericin, xanthan gum, alginate, gelatine, poly(sulfopropyl methacrylate), poly(acrylic acid), poly(methacrylic acid), poly(maleic acid-alt-methylvinyl ether), and poly(maleic acid-alt-styrene). In one embodiment, the polymer is poly(L-lysine) (PLL).

ELPs and ECM proteins (such as fibronectin, vitronectin, collagen, laminin, agrin, fibroin, elastin, or fragments or combinations thereof) are examples of polymer layers that can serve as the protein layer, and as such if a layer of such proteins is present (or a layer of an alternative protein that is able to adhere to adherent cells) then no additional polymer layers are needed (although they may be present).

Non-peptide polymers may be selected from the group consisting of poly(L-lysine) (PLL), poly(allylamine), poly(vinyl alcohol) (PVA), poly(hydroxyethyl methacrylate) (PHEMA), chitosan, poly(serine), poly(styrene sulfonate), chondroitin sulfate, hyaluronic acid, graphene oxide, polysaccharides (such as dextran, heparin, carboxy methyl cellulose, xanthan gum and alginate), poly(sulfopropyl methacrylate), poly(acrylic acid), poly(methacrylic acid), poly(maleic acid-alt-methylvinyl ether), and poly(maleic acid-alt-styrene). If a non-peptide polymer is used, an additional protein/peptide layer is required.

The polymers can be a copolymer or a block copolymer.

In some embodiments, the system may comprise a layer of graphene oxide (GO). For example, the polymer layer may comprise GO. The polymer may comprise a composite of PLL and GO. The composite may comprise multiple layers of PLL and GO. Such a composite may have a strengthening effect.

Multiple protein layers may be present. For example, even if the polymer layer is provided by a proteinaceous polymer, an additional protein layer may still be present.

When ELPs are used, they may be positively charged or negatively charged. In one particular embodiment, the positively charged ELP is (VPGIG VPGIG VPGKG VPGIG VPGIG)₂₄. In one particular embodiment, the negatively charged ELP is MESLLP-[(VPGVG VPGVG VPGEG VPGVGVPGVG)10-(VGIPG)60-V].

In one embodiment of the invention, the amount of the polymer may be determined according to the concentration of the polymer in the aqueous phase when they are deposited. A suitable concentration may be at least about 1 μg/ml, for example from about 1 μg/ml to about 100 mg/ml (weight of polymer in the corresponding volume of aqueous phase).

### Protein/Peptide Component of the Conditioning Layer

The protein/peptide layer of the conditioning layer (herein also referred to as the protein layer or the peptide layer of the conditioning layer) is situated at the top or outermost layer of the conditioning layer and is therefore disposed at the interface with the aqueous layer. The protein layer provides the support for culturing the cells at the liquid-liquid interface. The protein/peptide layer may be separate to the polymer layer (for example if the polymer layer is non-peptidic), or the protein/peptide layer may also serve as the or a polymer layer. The protein/peptide layer facilitates the adherence of the adherent cells.

In some embodiments, the protein layer comprises an extra-cellular matrix (ECM) protein or macromolecule mimicking the cell adhesive properties of ECM proteins. Relevant ECM proteins include: fibronectin, vitronectin, collagen, laminin, agrin, fibroin and elastin. The macromolecular mimic thereof is functionalised to provide cell adhesive properties. Natural ECM proteins are inherently presenting cell adhesive peptidic domains. Cell adhesive peptide sequences include RGD, YIGSR, IKVAV and PHSRN or other sequences that can bind integrin receptors.

Therefore, in one embodiment, the protein layer comprises a protein selected from the group consisting of extra-cellular matrix (ECM) proteins and macromolecules mimicking the cell adhesive properties of ECM proteins, wherein the macromolecule comprises a cell adhesive peptide sequence, for example a cell adhesive peptide sequence selected from the group consisting of RGD, YIGSR, IKVAV and PHSRN.

In some embodiments, the ECM protein is a protein selected from the group consisting of fibronectin, laminin, collagen, vitronectin, agrin, elastin and fibroin and functional fragments thereof. In preferred embodiments, the protein comprises collagen or fibronectin. In more preferred embodiments, the protein layer consists of collagen and/or fibronectin.

In some embodiments, the or a protein layer may be provided by serum. This may be separate to any serum or serum components present as part of the aqueous cell culture medium.

In a functioning cell culture system of the invention, the protein layer may be crosslinked (covalently or otherwise). Crosslinking, if present, takes place after assembly of the conditioning layer. In some embodiments, the protein layers are crosslinked by the bonding to the surfactant. For example, use of a surfactant that comprises sebacoyl chloride may bond with the protein layer to provide a covalently crosslinked protein layer.

In one embodiment of the invention, the amount of the protein/peptide may be determined according to the concentration of the polymer in the aqueous phase when they are deposited. A suitable concentration may be at least 1 μg/ml, for example from 1 μg/ml to 100 mg/ml (i.e. the weight of polymer in the corresponding volume amount of aqueous phase in the cell culture system).

Possible combinations of surfactant, oil, polymer and peptide/protein are as follows (this list is non-exhaustive):

| Surfactant | Oil | Polymer(s) | Protein(s) | Scenario (above) |
|---|---|---|---|---|
| pentafluorobenzoyl | Novec | PLL | Fibronectin (FN) | (c) |
| chloride (PFBC) | 7500 | | | |
| pentafluorobenzoyl | Novec | PLL and poly(sodium | Collagen (e.g. | (e) |
| chloride (PFBC) | 7500 | 4-styrenesulfonate) | collagen type I) | |
| pentafluorobenzoyl | Novec | PLL and GO | Fibronectin (FN) | (e) |
| chloride (PFBC) | 7500 | | | |
| Octanoyl chloride | PDMS | PLL | Fibronectin (FN) | (c) |
| Octanoyl chloride | PDMS | PLL and poly(sodium | Collagen (e.g. | (e) |
| | | 4-styrenesulfonate) | collagen type I) | |
| Sebacoyl chloride | PDMS | PLL | Fibronectin (FN) | (c) |
| Sebacoyl chloride | PDMS | PLL and poly(sodium | Collagen (e.g. | (e) |
| | | 4-styrenesulfonate) | collagen type I) | |
| pentafluorobenzoyl | Novec | ELP (for example a | Fibronectin (FN) | (c) |
| chloride (PFBC) | 7500 | positively charged ELP) | | (note polymer |
| | | | | layer is a protein) |
| pentafluorobenzoyl | Novec | PLL and ELP (for | Collagen (e.g. | (e) |
| chloride (PFBC) | 7500 | example a negatively | collagen type I) | (note one polymer |
| | | charged ELP) | | layer is a protein) |
| pentafluorobenzoyl | Novec | ELP (for example an | No additional | (a) |
| chloride (PFBC) | 7500 | ELP functionalised | protein necessary. | |
| | | for cell adhesion) | | |
| pentafluorobenzoyl | Novec | PLL and hyaluronic | Collagen (e.g. | (e) |
| chloride (PFBC) | 7500 | acid | collagen type I) | |
| pentafluorobenzoyl | FC-40 | PLL | Fibronectin (FN) | (c) |
| chloride (PFBC) | | | | |
| pentadecafluorooctanoyl | Novec | PLL | Fibronectin (FN) | (c) |
| chloride | 7500* | | | |
| Heptadecanoyl chloride | PDMS | PLL | Fibronectin (FN) | (c) |
| Heptadecanoyl chloride | PDMS | PLL | Fibronectin (FN) | (c) |
| and Sebacoyl chloride | | | | |
| (for example in a 3:1 | | | | |
| mixture) | | | | |
| Heptadecanoyl chloride | Rapeseed | PLL | Fibronectin (FN) | (c) |
| | oil | | | |
| Heptadecanoyl chloride | Rapeseed | PLL | Fibronectin (FN) | (c) |
| and Sebacoyl chloride | oil | | | |
| (for example in a 1:1 | | | | |
| mixture) | | | | |
| Heptadecanoyl chloride | Mineral | PLL | Fibronectin (FN) | (c) |
| and Sebacoyl chloride | oil | | | |
| (for example in a 1:1 | | | | |
| mixture) | | | | |

*Alternatively, the fluorinated oil can be, for example, FC-40.

In one embodiment of the invention, the cell culture system comprises:
a) an oil selected from the group consisting of a hexane,3-ethoxy-1,1,1,2,3,4,4,5,5,6,6,6-dodecafluoro-2-(trifluoromethyl), FC40 and PDMS;
b) a surfactant selected from the group consisting of PFBC, pentadecafluorooctanoyl chloride, octanoyl chloride, sebacoyl chloride and heptadecanoyl chloride, or a combination thereof; and
c) a polymer selected from the group consisting of PLL, poly(sodium 4-styrenesulfonate), ELP and hyaluronic acid; and optionally
d) a protein selected from the group consisting of collagen and fibronectin, or a combination thereof.

Component (b) may be present in an amount of from about 0.001 mg/ml to about 0.05 mg/ml, or from about 0.00125 mg/ml and about 0.01 mg/ml as measured with respect to the total volume of oil. Components (c) and (d) may each be present in an amount of from about from about 1 μg/mL to about 100 mg/ml as measured with respect to the total volume of the aqueous phase.

In more specific embodiments, the cell culture systems include the following combinations, which are presented as representative examples are not to be considered as limited on the scope of the invention:
Example suitable cell culture system components for HPKs (and other cell types):
   Fluorinated oil (Novec 7500)+Pentafluorobenzoyl chloride+
      1) PLL (100 μg/mL)+FN (10 μg/mL)
      2) PLL (100 μg/mL)+poly(sodium 4-styrenesulfonate) (MW: 1000,000) (100 μg/mL)+collagen type 1 (20 μg/mL)
      3) PLL (100 μg/mL)+poly(sodium 4-styrenesulfonate) (MW: 70,000) (100 μg/mL)+collagen type 1 (20 μg/mL)
      4) PLL (100 μg/mL)+GO (100 μg/mL)+PLL (100 μg/mL)+GO (100 μg/mL)+PLL (100 μg/mL)+GO (100 μg/mL)+PLL (100 μg/mL)+FN (10 μg/mL)
   PDMS+Octanoyl chloride+
      1) PLL (100 μg/mL)+FN (10 μg/mL)
      2) PLL (100 μg/mL)+poly(sodium 4-styrenesulfonate) (MW: 70,000) (100 μg/mL)+collagen type 1 (20 μg/mL)
   PDMS+Sebacoyl chloride+
      1) PLL (100 μg/mL)+FN (10 μg/mL)
      2) PLL (100 μg/mL)+poly(sodium 4-styrenesulfonate) (MW: 70,000) (100 μg/mL)+collagen (20 μg/mL)
Example suitable cell culture system components for MSCs (and other cell types):
   Fluorinated oil (Novec 7500)+Pentafluorobenzoyl chloride
      1) PLL (100 μg/mL)+FN (10 μg/mL)
      2) PLL (100 μg/mL)+poly(sodium 4-styrenesulfonate) (MW: 1000,000) (100 μg/mL)+collagen type 1 (20 μg/mL)
      3) PLL (100 μg/mL)+poly(sodium 4-styrenesulfonate) (MW: 70,000) (100 μg/mL)+collagen type 1 (20 μg/mL)
      4) PLL (100 μg/mL) and BSA (100 mg/mL) mixture+FN (10 μg/mL)
      5) PLL (100 μg/mL) and BSA (33.3 mg/mL) mixture+FN (10 μg/mL)
      6) ELP (+) (elastin like protein, positively charged, (VPGIG VPGIG VPGKG VPGIG VPGIG)24)+FN (10 μg/mL)
      7) PLL (100 μg/mL)+ELP(−) (elastin like kprotein, negatively charged, MESLLP-[(VPGVG VPGVG VPGEG VPGVGVPGVG)10-(VGIPG)60-V]) (100 μg/mL)+collagen type 1 (20 μg/mL)
      8) ELP-RGD, elastin like protein, positively charged, containing RGD, [[(VPGIG)2(VPGKG)(VPGIG)2]2AVTGRGDSPASS[(VPGIG)2(VPGKG)(VPGIG) 2]2]6 (100 μg/mL)
      9) PLL (100 μg/mL)+Hyaluronic acid (MW: 700,000) (100 μg/mL)+collagen type 1 (20 μg/mL)
      10) PLL (100 μg/mL)+Hyaluronic acid (MW: 60,000) (100 μg/mL)+collagen type 1(20 μg/mL)
   PDMS+heptadecanoyl chloride+PLL (100 μg/mL)+FN (10 μg/mL)
   PDMS+sebacoyl chloride+heptadecanoyl chloride+PLL (100 μg/mL)+FN (10 μg/mL)
   Rapeseed oil+heptadecanoyl chloride+PLL (100 μg/mL)+FN (10 μg/mL)
   Rapeseed oil+sebacoyl chloride+heptadecanoyl chloride+PLL (100 μg/mL)+FN (10 μg/mL)
   Mineral oil+sebacoyl chloride+heptadecanoyl chloride+PLL (100 μg/mL)+FN (10 μg/mL)

The polymer layers are layered onto the oil of the cell culture system to provide the conditioning layer, wherein the conditioning layer comprises at least one protein/peptide layer. In some embodiments, at least two layers of polymer are layered on to the surface of the oil, wherein the top layer is a protein/peptide layer. In some embodiments, at least two layers of polymer are layered on to the surface of the oil, wherein the top layer is a protein/peptide layer and the remaining layers are non-peptidic polymers. The surfactant may be present as a polymer layer, and said surfactant polymer layer is the bottom layer in the conditioning layer and is bonded to its adjacent polymer layer. If the surfactant is not a polymer surfactant, said surfactant is present with the bottom layer of polymer and is bonded to it. Optionally, the peptide/protein layer is crosslinked, for example the peptide/protein layer is crosslinked after the preceding layers (if any) have been layered onto the surface of the oil.

### Other Components of the Conditioning Layer

The pH of the conditioning layer can be controlled using normal methods (buffers etc.) to optimise the cell adherence and mechanical properties of the conditioning layer. In one embodiment of the invention, the pH of the conditioning layer is from about 9 to about 11 (for example pH 10.4). However, the pH of the aqueous phase of the cell culture system is a pH suitable for the culture of cells (for example between 7 and 8, optimally pH 7.4).

In some embodiments of the invention, the conditioning layer of cell culture system may be further optimised for the adherence of cells. For example, the top layer of the conditioning layer may comprise a ligand or antibody that adheres to a cell of interest. Examples of suitable ligands or antibodies include integrin receptors (RGD, YIGSR, PHSRN, IKVAV) or antibodies against cadherins.

### Types of Cells to be Cultured

The cell culture systems and methods of the invention are suitable for the culture of adherent cells. The systems and methods of the invention are particularly suited for the culture of stem cells, more particularly adherent stem cells.

In some embodiments the cells will be human cells, although other cell types can also be used with the invention (in particular mammalian cells, such as equine, canine, porcine, bovine, ovine, or rodent (e.g., mouse or rat) cells).

In one embodiment of the invention, the cells to be cultured are selected from the group consisting of human primary keratinocytes (HPKs), mesenchymal stem cells (MSCs), induced pluripotent stem cells (iPSCs), Chinese hamster ovary (CHO) cells, human umbilical vein endothelial cells (HUVECs), adipose derived stem cells, amniotic fluid derived stem cells, hepatocytes, lung epithelial cells, cord blood stem cells, fibroblasts and cardiomyocytes, although the cell culture systems are not limited to the culture of these cell types.

In one embodiment of the invention, the cells are adherent stem cells. The adherent stem cells may be selected from the group consisting of Human Primary Keratinocytes (HPKs), mesenchymal stem cells (MSCs), induced pluripotent stem cells (iPSCs), adipose derived stem cells, amniotic fluid derived stem cells and cord blood stem cells.

In one embodiment of the invention, the cells are human primary keratinocytes.

In another embodiment of the invention, the cells are human mesenchymal stem cells. Mesenchymal stem cells (MSCs; alternatively known as mesenchymal progenitor cells) are cells capable of expanding in culture and differentiating into mesenchymal tissue cells, including bone, cartilage, tendon, ligament, muscle, adipose, and marrow stroma.

In one embodiment of the invention, the cells are induced pluripotent stem cells (iPSCs). Induced pluripotent stem cells can be directly generated from adult cells, can propagate indefinitely, and are capable of differentiating into any cell type in the body.

### Properties of the Conditioning Layer

An additional and important consideration of the selection of the components on the cell culture system are the mechanical properties of the resulting cell culture support at the liquid-liquid interface. The present inventors have surprisingly found that the cell culture systems of the invention provide a surface having a suitable elasticity that enables the long-term culture of adherent cells, including stem cells. The investigations by the inventors surprisingly found that cell proliferation is better correlated with the level of elasticity (stress retention) than with maximum stress (stiffness) of the conditioning layer. The combinations of surfactant, protein/peptide layer and optional additional polymers used in the present invention to form the conditioning layer provide a rigid nanoscale quasi 2D-material that can support the culture of adherent cells.

An important mechanical property of the cell culture systems is the elasticity of the liquid-liquid interface. The elasticity of this interface is controlled by the conditioning layer containing the surfactant. The elasticity of the interface is sufficient to enable adherent cells (such as adherent stem cells) to proliferate to at least about 50% confluency. The measure of elasticity is a measure of the degree of elasticity of the interface (including the surfactant-polymer-protein film assembled at the interface) between the aqueous and oil phases of the cell culture system.

In one embodiment of the invention, the elasticity (i.e. stress retention) is at least about 60%, or at -preferably at least about 65%. When elasticity is at least about 65%, the culture of dense adherent cell colonies is possible even without acto-myosin inhibitors, such as the ROCK inhibitor. Accordingly, the cell culture systems of the invention do not include or require any acto-myosin inhibitors.

Elasticity can be measured according to any suitable method known to the skilled person. One such method is the use of a rheometer. For example, one method may use a rheometer fitted with an interfacial rheology system to allow stress-relaxation experiments to be carried out. In such set up, a Du Noüy ring is fitted to the shaft of the rheometer and oscillates. The percentage elasticity can be defined as the level of stress retained at infinite time (extracted from curve fitting), compared to the stress exerted on the sample just before relaxation is allowed to start. Similar experiments can be carried out with an oscillating magnetic bar positioned at the interface between the two liquids, using a magnetic rig to monitor deformations.

Accordingly, in one embodiment, the elasticity of the liquid-liquid interface is at least about 65% as measured in a stress-relaxation experiment using a rheometer. In another embodiment of the invention, the elasticity of the liquid-liquid interface is at least about 65% as measured using a Du Noüy ring tensiometer.

One detailed method to quantify interfacial stress relaxation is as follows. Rheological measurements were carried out on a hybrid rheometer (DHR-3) from TA Instruments fitted with a double wall ring (DWR) geometry and a Delrin trough with a circular channel. The double wall ring used for this geometry has a radius of 34.5 mm and the thickness of the Platinum-Iridium wire is 1 mm. The diamond-shaped cross-section of the geometry's ring provides the capability to pin directly onto the interface between two liquids and measure the interface properties without complicated sub-phase correction. 19 mL of the fluorinated oil pre-mixed with surfactant were placed in the Delrin trough and the ring was lowered, ensuring contact with the surface, via an axial force procedure. The measured position was set 500 μm lower than the contact point of the ring with the oil-phase surface. Thereafter, 15 ml of the PBS buffer were carefully syringed on top of the oil phase. Stress relaxation tests were performed during 120 s, with a strain rate of 0.5%/s and 1%/s, for maximum strains of 0.5% and 1%, respectively.

After locating the maximum stress, the stress relaxation data was fit to a double exponential decay function:

$$y = A 1 \times exp ( - \frac{x}{t 1} ) + A 2 \times exp ( - \frac{x}{t 2} ) + y 0 .$$

Stress retention (in % of the maximum stress) was calculated by equation:

$$Stress retention \% = \frac{y 0}{y 0 + A 1 + A 2} \times 100 \%$$

The thickness of the conditioning layer can also influence its suitability for culturing adherent cells. In one embodiment, the dry thickness of the conditioning layer is from about 1 nm to about 10 μm, from about 1 nm to about 100 nm, from about 1 nm to about 50 nm or from about 1 nm to 20 nm. The thickness is the dry thickness of the layer and can be measured by atomic force microscopy. Swollen thicknesses can be measured using interometry, 3D optical profiling, neutron reflectivity or ellipsometry.

In one embodiment of the invention, the shear interfacial modulus of the interface is at least about 0.01 N/m.

In one embodiment of the invention, the elasticity of the liquid-liquid interface is at least about 65%, the shear interfacial modulus of the interface is at least about 0.01 N/m, and the dry thickness of the conditioning layer is from about 1 nm to 20 nm. In another embodiment of the invention the elasticity of the liquid-liquid interface is at least about 65%, the shear interfacial modulus of the interface is at least about 0.01 N/m, and the dry thickness of the conditioning layer is from about 10 nm to 20 nm

### Aqueous Cell Culture Medium

The aqueous cell culture medium can be any suitable cell culture medium in the art useful in the culture of adherent cells, and the skilled person will be aware of appropriate cell culture media and will be able to choose an appropriate cell culture media for the culture of a given population of cells. The choice of cell culture medium will be familiar to the person of skill in the art and will depend on the type of cells being cultured. Advantageously, the cell culture systems of the invention can use the same cell culture medium as would be used for a given cell type if it was being grown on a solid substrate.

For example, the aqueous cell culture media may comprise a carbon source, various salts and optionally a source of amino acids and/or nitrogen. The media may be a chemically defined media (in which all of the components or known), or an undefined culture media may be used, which comprise yeast, animal or plant extracts (such as BSA).

Depending on the context, the cell culture system may comprise selective media, differential (i.e. differentiation-inducing) media, transport media, cell-sorting media, or enriched media. Again, the appropriate choice of media would be apparent to the skilled person for a given context (cell type, assay, length of culture etc. Examples of suitable cell culture media for the culture of adherent cells, including stem cells, include DMEM (many different cell types), FAD (for example for keratinocytes), KSFM (for example for keratinocytes) and EBM-2 (for example for HUVECs), and there are many more available that could be used in the cell culture systems of the invention.

The aqueous cell culture medium may be sterile. When culturing cells, the cell culture medium may be replaced or replenished to allow the longer-term culture of cells.

In one aspect of the invention, the cell culture system is suitable for use in a bioreactor, in particular a 3D bioreactor. The bioreactor contains the cell culture system of the invention and a culture of adherent cells, such as adherent stem cells. The cells adhere to the liquid-liquid interface. The bioreactor can take any suitable form, for example the bioreactor may be a cell culture flask or bag.

### Methods of Culturing Cells

The present invention provides a method of culturing adherent cells comprising culturing the cells in a cell culture system of the invention. Methods of culturing cells is also referred to herein as a method of expanding a cell population, since a cultured cell population will be expanded by the method. The cell cultures are not suspension cultures as the cells are adhered to the conditioning layer of the cell culture system.

Such methods of the invention may comprise seeding the cells at the liquid-liquid interface of the cell culture system (in particular, at the protein layer of the conditioning layer of the system) and contacting the seeded cells with a cell culture medium. The choice of cell culture medium will be particular to the cells being cultured and the skilled person is familiar with which cell media are suitable for which cell types.

In some embodiments of the invention, the cells are cultured for at least about 1 day, or at least about 5 days, or at least about 7 days, or at least about 14 days. In preferred embodiments, the cells are cultured for at least about 7 days. Advantageously, the cell cultures systems of the present invention are suitable for this long-term culture of adherent cells, including stem cells. The cell culture systems allow the culture of cells over several days (for at least about 1 day, or at least about 5 days, or at least about 7 days, or at least about 14 days) wherein over 80% cells remain alive.

The cells may be cultured until they reach confluency. For example, the cells may be cultured for a sufficient time for the cells to reach at least 50% confluency. The cell culture systems of the invention surprisingly allow the culture of adherent cells (including adherent stem cells) to at least 50% confluency even when culturing the cells at a liquid-liquid interface, or to at least 4 times the initial cell population (or preferably to at least 10 times the initial cell population).

In some embodiment of the invention, the method comprises harvesting the cultured cells or cell sheets from the cell culture system. This can be achieved by any suitable method, for example centrifugation (for example at 1200 rpm for 5 min, but not exclusively), by transferring cells to another substrate, by allowing the oil to evaporate, or by using a chemical or enzyme that allows to partially degrade the protein and/or protein that are present at the interface.

One of the advantages of the present invention is that the methods used to harvest the cells do not require enzymatic digestion or treatment at low temperatures, which are significant drawbacks of current culture methods that grow cells on solid substrates.

Alternatively, the cultured stem cells can be used without being harvested from the cell culture system. For example, when the cell culture system is in the form of an emulsion, the method may comprises administering the emulsion containing the cultured cells directly to a patient or to a tissue engineering platform. In one embodiment of the invention, the method comprises administering a culture of cells to a hydrogel. Such embodiments are useful for tissue engineering such as 3D tissue engineering.

### Methods of Preparation of the Cell Culture Systems

The present invention also provides a method of production of the cell culture systems of the invention. The cell culture systems are manufactured such that the assembly of the protein and the polymer layer at the interface between the two phases is mediated by the surfactant. These can be assembled by simply placing in contact the oil phase and the aqueous phase (including suitable surfactant, polymers and/or proteins) and agitating vigorously to create an emulsion. Alternatively, this can be done in a more controlled way using a microdroplet or picodroplet fabrication method or other similar systems allowing the formation of emulsions.

In one embodiment of the invention, the method of production of the cell culture system comprises contacting the chosen oil, surfactant and protein/peptide with the aqueous medium and forming an oil-in-water emulsion. If using a separate polymer, this may also be included. The step of forming an oil-in-water emulsion may comprise, for example, shaking the mixture containing the components of the cell culture system. Alternatively, the emulsion may be formed using microdroplet or picodroplet fabrication platforms. In embodiments where multilayers of polymers/proteins are used, the layered components are introduced sequentially after washing of the first aqueous phase and introduction of a new aqueous phase introducing suitable polymers and proteins. For example, the method may comprise contacting the chosen oil and components of the first layer of the conditioning layer with a first aqueous medium and forming an oil-in-water emulsion (in multi-layered embodiments, shaking, stirring or microdroplet formation can be used for the first layer, but for subsequent layers simple layer-by-layer deposition may be a preferred method). Subsequently, the first aqueous medium is removed (for example by washing), and the oil emulsion is contacted with the component or components of the second layer of the conditioning layer and a second aqueous medium (which may be the same as the first aqueous medium). This is repeated until all the layers of the conditioning layer have been formed around the oil.

Alternatively, 2D interfaces can be generated by sequential incubation and washing steps (no step of forming an emulsion is required when the cell culture system is a sheet). Accordingly, in one embodiment of the invention, the method comprises contacting the chosen oil and components of the first layer of the conditioning layer with a first aqueous medium and incubating the components to allow a planar interface to be formed between the two components. The cell culture system is then washed before contacting the system with the component or components of the second layer of the conditioning layer and a second aqueous medium (which may be the same as the first aqueous medium) followed by a further incubation step. This is repeated until all the layers of the conditioning layer have been formed on the surface of the oil There is also provided a kit of parts comprising a surfactant, an oil and a peptide/protein as defined herein for the culture of adherent cells. The surfactants, oils and proteins are the surfactants, oils and proteins useful for the culture of adherent cells according to a method of the invention. In one embodiment, the kit further comprises an additional polymer for inclusion in the cell culture system.

The components of the kit will generally be disposed separately. For example, each of the oil, surfactant, and peptide/protein (and polymer if using) are disposed in separate containers. In one embodiment, the kit further comprises instructions for use (for example instructions for the manufacture of the cell culture system and/or for the culture of adherent cells according to a method of the invention).

The present invention provides populations of cells that have been cultured or expanded according to a method of the invention

### Additional Embodiments of the Invention

The invention further provides the use of the cultured and/or expanded populations of adherent cells in medicine.

For examples, cells grown according to the methods of the invention may be useful in tissue engineering, such as bone regeneration, wound healing, cartilage regeneration, tendon regeneration and cardiac repair (for example, in the treatment of myocardial infarction). The desired cells, in particular stem cells, can be cultured according to the method of the invention, including possible differentiation of the cells, harvested, and then applied to a patient in a suitable manner, such as in the form of a bioengineered tissue construct. The tissue construct can be formed into an appropriate shape or arrangement according to its purpose. For example, in the case of cells grown in sheets according to a method of the invention, said cell sheets may be applied directly. Alternatively, cells cultured according to methods of the present invention may be applied to a 3D scaffold. Suitable scaffolds are biocompatible and may be biodegradable such that the scaffold is slowly degraded after implantation into a patient. The scaffolds will have mechanical properties that are suitable for the intended purpose. Suitable scaffolds include hydrogel or collagen scaffolds. Suitable tissue engineering techniques that employ scaffolds are discussed in, for example, O'Brien, Materials Today, 14(3):88-95, 2011.

Cells cultured according to methods of the invention may be used for the generation of proteins, such as growth factors, cytokines, therapeutic peptides, microRNA or antibodies.

The present invention therefore also provides a method of producing a protein or other molecule of interest, comprising culturing a cell according to a cell culture method of the invention, wherein the cell expresses or produces the protein or molecule of interest, and collecting the protein or molecule of interest from the cell culture medium. The cells may have been transfected or otherwise engineered to product the protein or molecule of interest. For example, the cells may have been transfected with vectors encoding for a protein of interest. The sequence encoding the protein is operable linked to a promoter that is compatible with the cell being transfected. In some embodiments of the invention, the method includes the step of transfecting the cell with the plasmid encoding the protein of interest.

For example, in the case of antibody production, a cell may be transfected with two plasmids, one plasmid encoding a heavy chain of an antibody and the other plasmid encoding a corresponding light chain of an antibody, wherein the sequences encoding the heavy and light chains are operably linked to promoters that compatible with the cell being transfected (such as a CMV promoter). The cells express the sequences encoding the antibodies and via post-translational modification secrete the assembled antibody into the cell culture medium. The antibody can then be extracted from the cell culture medium in the usual way.

An appropriate cell type can be used to generate the protein or other molecule of interest. For example, although the methods of the invention are particularly suited for the culture of stem cells, other adherent cells may be useful in the contest of the generation of proteins (such as antibodies). CHO cells may be of particular use for this purpose.

The invention also provides various methods of treatment using cell populations cultured or expanded according to a method of the invention. For example, the invention is useful in the expansion of stem cell populations in stem cell therapy, including allogenic and autologous stem cell therapy.

In one embodiment of the invention, there is provided a method of culturing a population of adherent stem cells according to a method described herein, obtaining an expanded population of cells, and administering the expanded population of cells to a patient. In autologous stem cell therapy, the donor and recipients of the cells are the same patient. The stem cells may be obtained by any suitable means known to the skilled person, for example the isolation of stem cells from a patient sample such as a patient's bone marrow. Once isolated, the cells can the washed and seeded onto a cell culture system of the invention and expanded until a suitable confluency is reached.

The present invention also provides methods for the purification of adherent cells, including adherent stem cells. In such cases, cell mixtures (e.g. obtained from bone marrow or adipose tissue aspirates) are directly placed in contact with oil emulsions functionalised with suitable surfactant/polymer/protein interfaces and briefly incubated (for example 20 min to 1 h) before separating the remaining cells from the emulsions (with bounded purified cells). This separation can be carried out via sedimentation or simple centrifugation (1200 rpm for 3-5 min).

Accordingly, in one embodiment of the invention there is provided a method of purifying adherent cells, comprising:
a) providing a mixture of cells obtained from a patient, wherein the mixture of cells comprises a cell population of interest;
b) contacting the cells with a cell culture system of the invention;
c) incubating the cells in the cell culture system;
d) separating cells of the cell population of interest from the remaining cells in the mixture.

The mixture of cells may be a cell sample from a patient that is obtained and requires purification to obtain a purified cell population of interest. The sample may be a sample that comprises stem cells. In one embodiment, the sample may be a bone marrow sample or an adipose tissue sample (for example a bone marrow or adipose tissue aspirate).

The step of contacting the cells with a cell culture system of the invention may comprise seeding the mixture of cells onto the cell culture substrate (i.e. the conditioning layer) of the cell culture system (i.e. the surface of the oil). Cell culture systems in the form of emulsions are of particular use in the methods of purification as they provide a larger surface area for capturing the cells of the cell population of interest and it is easier to mix the contents of the patient sample with emulsions (for example, they can be mixed by simple shaking).

The step of incubation can be carried out for a sufficient time to allow the cells to adhere to the conditioning layer of the cell culture system. For example, the step of incubation may comprise incubation of the cells in the cell culture medium for at least 10 minutes. This allows cells of the cell population of interest to adhere to the conditioning layer.

The step of separating the cells of the cell population of interest from the remaining cells in the mixture may comprise removing the oil with conditioning layer and adhered cells from the cell culture system. This can be achieved by, for example, centrifugation or sedimentation (or other suitable methods known to the skilled person). In this way, a purified population of cells can be provided.

The method may further comprise a step of culturing the captured cells in the cell culture system. This enables the population of adhered cells to expand. Of course, the cells can be later harvested from the cell culture system as well, as described elsewhere.

The cell population of interest is an adherent cell population. The cell population of interest may be a population of stem cells. For example, if the sample was an adipose tissue sample or a bone marrow sample, the cell population of interest may be a population of mesenchymal stem cells.

Purification of cells may be further improved by including a ligand or antibody that promotes adhesion of cells belonging to the cell population of interest. Example such ligands include protein A or protein G, or a combination thereof, optionally with albumin. The ligand or antibody may be incorporated into the cell culture system when the cell culture system is manufactured, to provide cell culture system that comprise the ligand for the cell population of interest. The ligand or antibody can be incorporated into the cell culture system by any suitable means, for example by directly adsorbing the ligand, adsorbing a first polymer layer (PLL or other cationic polymer, for example), followed by adsorption of the ligand, or by forming a biotinylated polymer layer (for example based on PLL-PEG-biotin), followed by streptavidin binding and capture of a biotinylated ligand.

In a more specific embodiment, the method of purification may comprise mixing the patient sample with a cell culture system of the invention that is an emulsion, for example by shaking. To promote adherence of the cell population of interest to the cell culture system, the emulsion may include a ligand that is specific for the desired cell type (for example an antibody that is specific to a cell surface marker present in the desired cell type). The emulsion comprising the adhered cells can then be separated from the remaining contents of the cell sample, for example by centrifugation, to allow the cell population of interest to be cultured using the cell culture system of the invention.

Methods of purification and culture of cells according to a method of the invention may promote the expression of stem cell markers in cultured stem cells or purified stem cell populations. The stem cells may also exhibit low levels of expression of differentiation markers (such as OCN and ALP). Without wishing to be bound by theory, the inventors hypothesise this is due to a selected effect of the culture of cells on non-flat liquid-liquid interfaces as stems cells with low stem cell surface markers (lower stem cell potential) are not able to adhere as efficiently to the liquid-liquid interface and therefore are selected out. Therefore, the methods of the invention provide a general and simple mechanism to sort cells, replacing other sorting technologies such as fluorescence activated cell sorting (FACS) and magnetic-activated cell sorting (MACS). In addition, it is hypothesised that selection of “better” stem cells can be achieved via initial adhesion of cells expression the highest levels of integrins and stem cell markers.

The present invention also provides the use of the cell culture systems of the invention in the purification of an adherent cell population.

In one embodiment of the invention, there is provided a method of culturing adherent stem cells in a cell culture system, the cell culture system comprising an aqueous cell culture medium and an oil phase in the form of an emulsion, wherein the oil is a fluorinated oil or silicone oil and is functionalised with a conditioning layer that comprises:
a) a non-polymeric surfactant and a polymer as a bottom layer, wherein the polymer is PLL and wherein the surfactant is bonded to the polymer layer via covalent and/or supramolecular forces; and
b) a protein layer as a top layer, wherein the protein is selected from the group consisting of fibronectin and collagen or a combination thereof;
wherein the bottom layer of the conditioning layer is adjacent to the oil phase and the top layer of the conditioning layer is adjacent to the aqueous phase;
and wherein the method comprises seeding a population of stem cells onto the conditioning layer of the cell culture system and culturing the stem cells in the cell culture system. The non-polymeric surfactant may be heptadecanoyl chloride, octanoyl chloride, sebacoyl chloride, perfluorooctanoyl chloride or PFBC or mixture of these surfactants. The stem cells may be HPKs or MSCs. The method may further comprise harvesting the cells from the cell culture medium after culture for at least 7 days, wherein at least 80% of the cells are alive.

Preferred aspects of the second and subsequence aspects of the invention are as provided for the first aspect of the invention, mutatis mutandis.

The present invention will now be further exemplified by reference to a number of specific examples, which are not intended to be limited on the scope of the invention.

### EXAMPLES

### Example 1

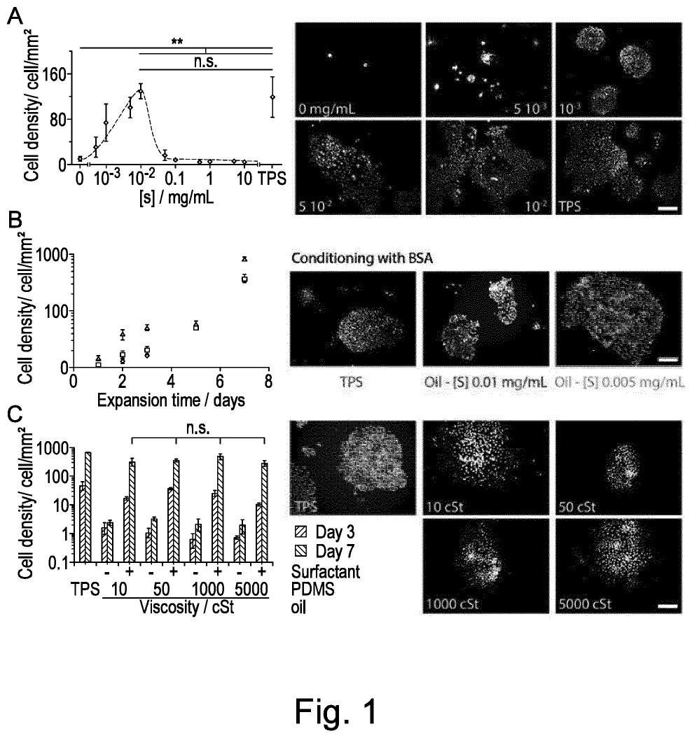

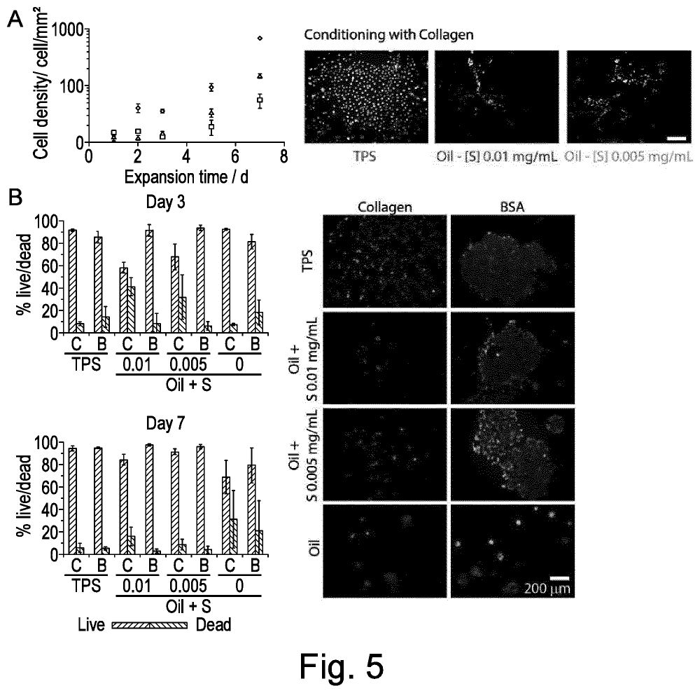

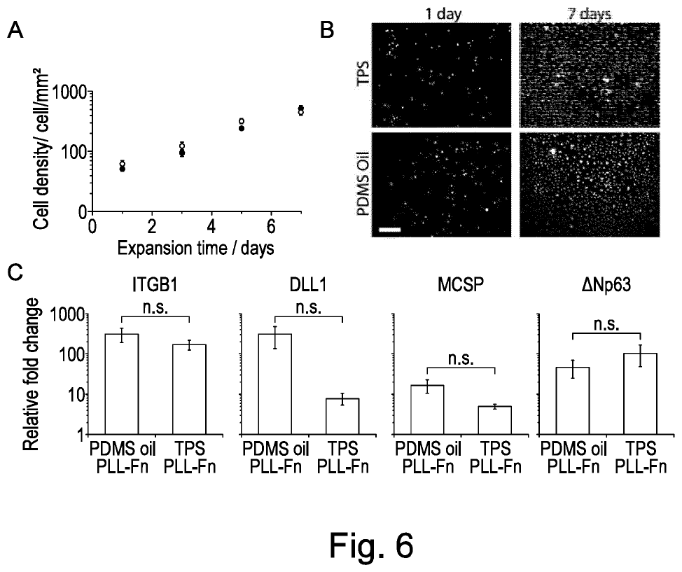

The present inventors have previously proposed that the nanoscale mechanics of the interface may dominate over bulk cues to regulate cell phenotype⁶. Indeed, stem cells did not respond to changes in the bulk modulus of silicones, over a very wide range (0.1 kPa to 2.3 MPa), in contrast to their behaviour at the surface of hydrogels. In addition, the inventors found that the softest silicones used (100 Pa) did not display any elasticity in stress relaxation experiments (FIG. 5), suggesting that cells may spread and proliferate on liquid substrates. To explore further the apparent lack of response of cells to the mechanical properties of silicones, the inventors seeded HaCaT cells on uncured, liquid silicone substrates (Sylgard, FIG. 1A). The inventors found that, even in the absence of a biofunctionalisation step, cells proliferated on this liquid silicone to comparable levels to those of tissue culture plastic. In order to test the role of substrate viscosity on cell behaviour, the inventors seeded HaCaTs on PDMS oils with viscosities ranging from 10 to 5000 cPs (FIG. 1A). Cells did not proliferate on these fluid substrates, in contrast to their behaviour on uncured Sylgard oil. The inventors proposed that components entering in the formulation of Sylgard-silicones may act as surfactants strengthening protein and cell adhesion. Indeed, the inventors observed that HaCaT cells proliferated at the surface of a fluorinated oil supplemented with a surfactant (pentafluorobenzoyl chloride) (FIG. 1B). The inventors found that the growth profile and viability of HaCaTs on fluorinated oil containing 10 and 5 μg/mL of surfactant and conditioned with bovine serum albumin (BSA) closely matched that of cells cultured on tissue culture plastic (TPS, FIG. 1C and FIG. 6). This was also observed in the case of interfaces conditioned with medium, but not those conditioned with collagen (FIG. 6). Such behaviour on protein-conditioned interfaces suggested that the surfactant-assisted adsorption of proteins to hydrophobic fluid interfaces is required to allow cell adhesion and proliferation.

### Example 2

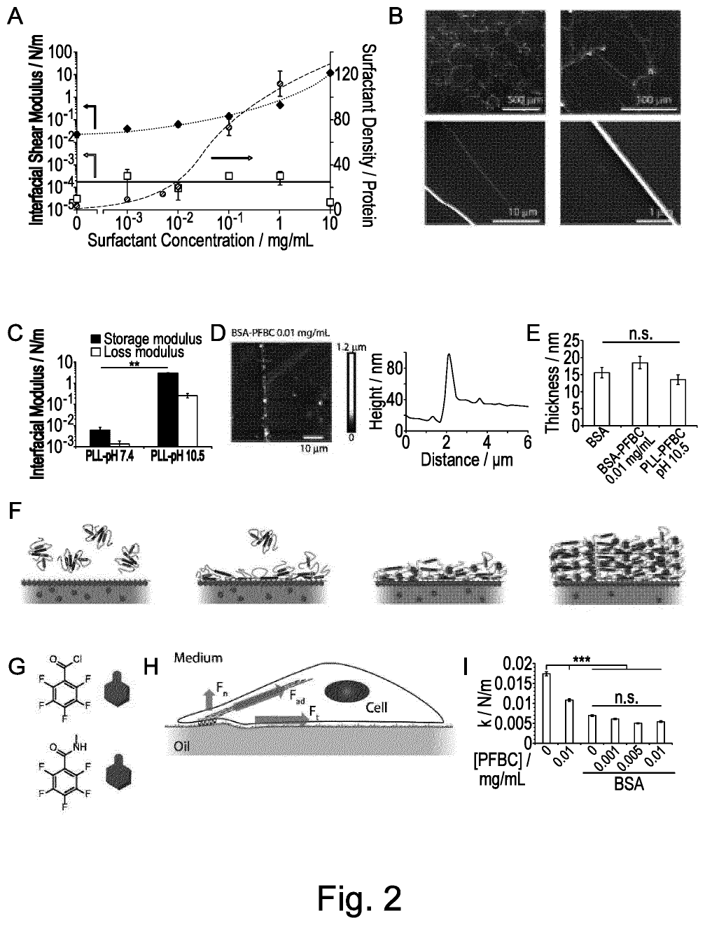

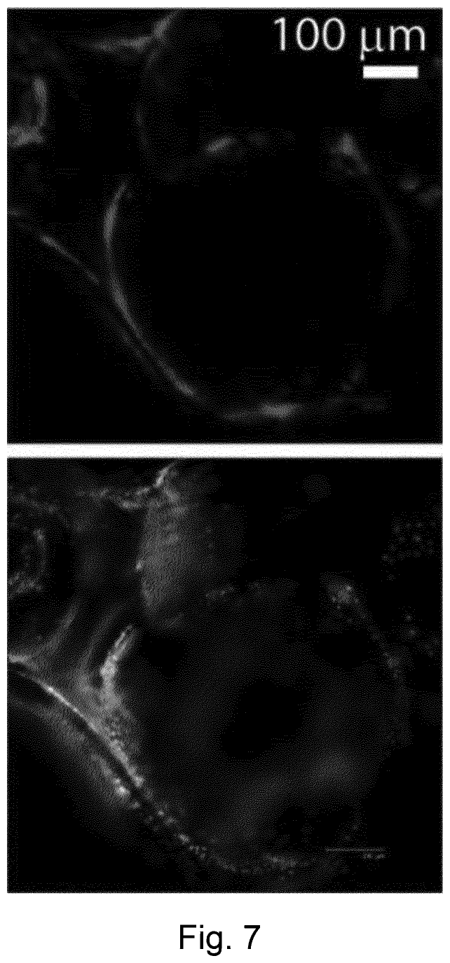

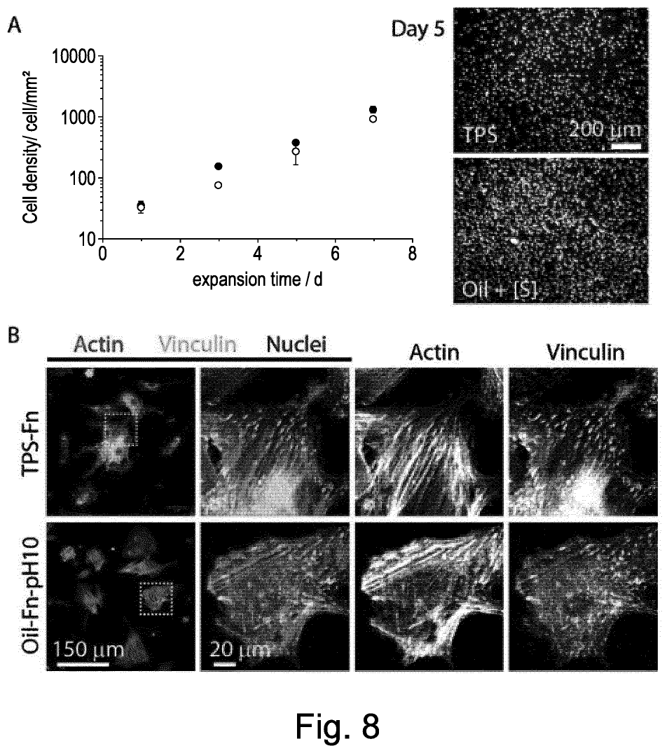

In order to investigate further the process of protein adsorption to oil interfaces, the inventors used interfacial rheology^{12,13} to monitor associated changes in shear mechanical properties at the oil/buffer interface (FIG. 7A). The interfacial shear storage modulus of oil-buffer interfaces remained low (10⁻⁵-10⁻⁴ N/m) and relatively insensitive to the surfactant concentration (FIG. 2A and FIG. 7B). Upon addition of BSA, the storage modulus increased by 2 to 5 orders of magnitude, depending on the surfactant concentration. The time sweep profile of such adsorption followed two main stages, in agreement with previous reports of protein adsorption at oil-water interfaces¹⁴⁻¹⁶: a sharp increase in the storage modulus in the first 15-20 min, corresponding to the adsorption of proteins to the interface, followed by a strengthening stage, during which the storage modulus continued to increase modestly. In the case of the highest surfactant concentration tested (10 mg/mL), a second sharp increase in interfacial mechanics was observed during this strengthening stage, corresponding to the formation of multiple protein layers. Such processes were found to depend on the protein type and concentration as well as the presence of surfactants (although often resulting in the displacement of proteins from the interface¹⁷). Importantly, the BSA films formed at the oil-water interface were clearly strengthened (from 10⁻²-10 N/m) as the surfactant concentration increased (FIG. 2A and FIG. 7C). This trend correlated with a gradual increase in the content of fluorinated surfactant bound to the protein layer (up to 112±11 surfactant/BSA molecule), as evidenced by XPS (FIG. 2A and FIGS. 8A and B). The presence of surfactants was also identified by FTIR, as protein assemblies displayed several bands in the region 1100-1250 cm⁻¹, corresponding to C-F stretching modes¹⁸ (FIG. 8C-E). In addition, FTIR provided evidence for the covalent coupling of surfactant to protein molecules, via the shoulder observed at 1720 cm⁻¹, corresponding to C═O stretching of esters. The shift in the amide I (1640-1660 cm⁻¹) and amide II (1520-1535 cm⁻¹) bands and the change in the ratio of their intensity indicated a reorganization of the protein structure and its unfolding at the oil surface¹⁹.

### Example 3

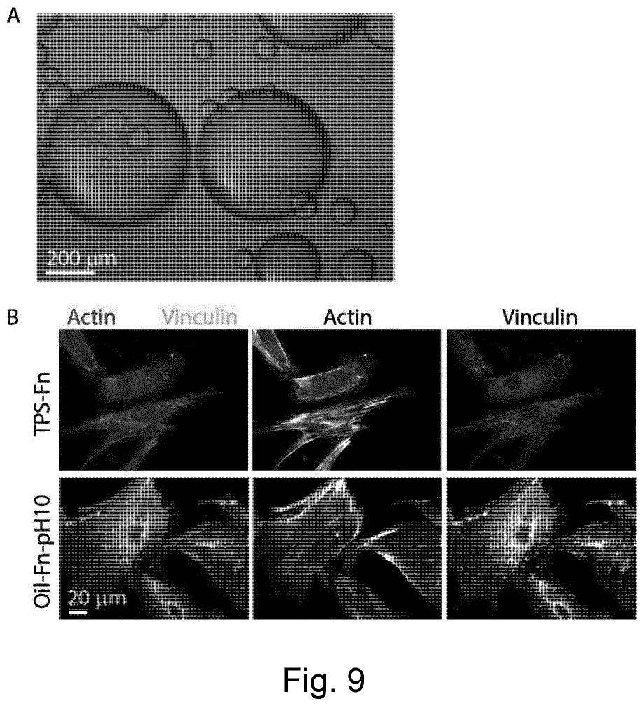

The thickness of protein assemblies was characterised to determine whether these structures remained quasi-2D sheets. Oil-in-buffer emulsions were deposited on silicon substrates and collapsed upon drying, leaving wrinkled skins corresponding to two proteins layers, as observed by SEM (FIG. 2C). The thickness of these protein sheets ranged from 14±2 to 19±2 nm, based on AFM characterisation (FIG. 2D and FIGS. 9A and B). SEM characterisation of wrinkles afforded thicknesses in the range of 36±5 to 57±12 nm, slightly higher than those measured by AFM as SEM overestimates the cross-section of the double layer (FIG. 9C-E). Overall, our results show that BSA assembles at fluorinated oil interfaces into partially denatured protein layers crosslinked by the incorporation of hydrophobic surfactants, resulting in the strengthening of the sheets and providing a suitable mechanical environment to sustain cell cycling.

### Example 4

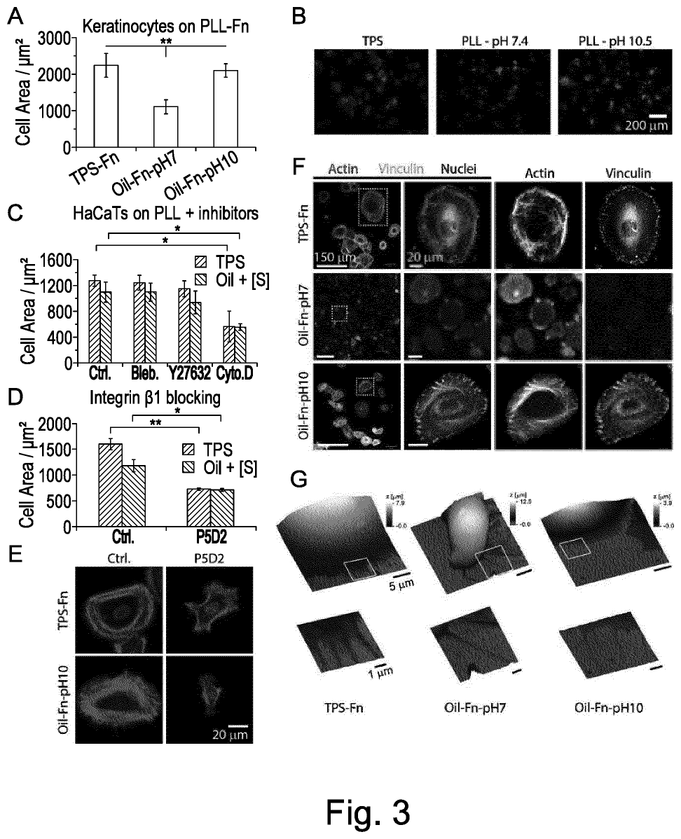

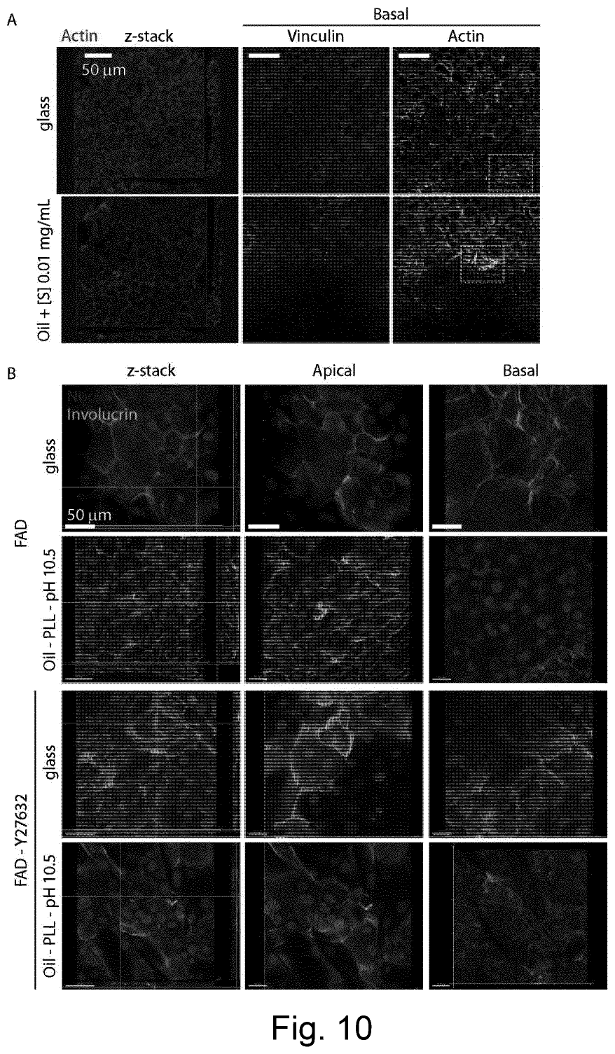

The mechanism via which HaCaT cells sense interfacial mechanics was investigated next. Since BSA is unlikely to directly act as ligand for integrin binding in HaCaTs, the inventors studied whether cell-cell adhesions could drive the phenomenon observed. Experiments carried out at a low Ca²⁺ concentration (<20 μM) showed that proliferation occurred in the absence of cell-cell adhesions (FIG. 10A), despite a 3-fold reduction compared to plastic control. Hence conditioning of the BSA interface by serum proteins is likely to contribute to the proliferation of HaCaT cells. The inventors found indeed that, although cell spreading was impaired on oils conditioned with medium, BSA and collagen, it still occurred following a delayed kinetics compared to TPS (FIG. 10B). To test the role of acto-myosin contractility in cell adhesion to liquid substrates, the inventors treated cells with blebbistatin, the ROCK inhibitor Y27632 and the actin depolymerizing drug Cytochalasin D (FIG. 3A and FIG. 10C). Blebbistatin led to a slight increase in cell spreading on oil, suggesting that myosin-based contractility limits cell spreading on deformable substrates. When exposed to the ROCK inhibitor Y27632 and cytochalasin D cell spreading further decreased, confirming the importance of the actin cytoskeleton in this process.

### Example 5

To improve the adsorption of ECM proteins and cell adhesion to oil interfaces, the inventors deposited first poly(L-lysine), followed by fibronectin adsorption (as is classical for the coating of glass substrates). Characterisation of the mechanical properties of the PLL layer generated confirmed the high modulus of the interface formed (see FIG. 3B and FIG. 7D). In addition, the inventors found that the pH of the buffer used during PLL adsorption had a major impact on the interfacial modulus of the layer generated (two orders of magnitude increase between pH 7.4 to 10.5, FIG. 3B), presumably due to the increased rate of surfactant coupling at higher pH (XPS indicated 13% functionalisation at pH 10.5, FIGS. 8A and B). The modulus obtained at pH 10.5 was comparable to that obtained with BSA at pH 7.4 with a considerably higher surfactant concentration (above 1 mg/mL, FIG. 2A). However, the thickness of the layer formed remained comparable to that measured for BSA interfaces (14±2 nm, FIG. 2D), corresponding to an extrapolated bulk shear modulus of 220±10 MPa (compared to 3.4±2.3 MPa for BSA interfaces). These results indicate that the high functionalisation level of PLL leads to the formation of a relatively hydrophobic and stiff polymer layer at the oil-buffer interface.

Cell spreading was affected by the mechanical properties of the PLL interfaces formed (FIGS. 3C and E). HaCaT spreading was improved when PLL adsorption was carried out at pH 10.5 compared to 7.4 (1070±20 μm² vs 780±40 μm²). Primary keratinocytes behaved similarly and their spreading on stiff PLL interfaces was very close to that of cells spreading on TPS (FIG. 3D). In addition, keratinocytes spreading on stiff PLL interfaces established a structured actin cytoskeleton, with stress fibres, and assembled focal adhesions, as on TPS, whereas cells adhering to soft PLL-oil interfaces did not display these structures (immunofluorescence microscopy showed instead actin rich protrusions reminiscent of the early stages of cell spreading^{5,20}, FIG. 3F). This was further confirmed by scanning ion conductance microscopy²¹, which demonstrated the formation of lamellipodia and filopodia at the surface of stiff, but not soft oil interfaces (FIG. 3G). Hence cells sense the nanoscale mechanics of liquid interfaces via an integrin-mediated mechanism, relying on the assembly of focal adhesions and a mature actin cytoskeleton.

### Example 6

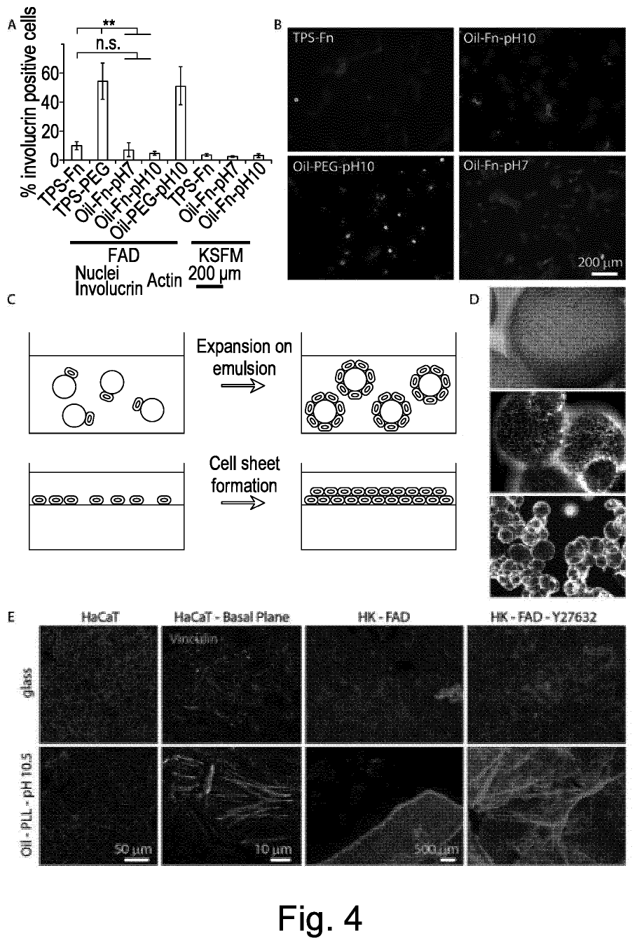

To establish the relevance of liquid substrates for stem cell expansion, the inventors examined the impact of liquid interfaces on stem cell fate. Bulk mechanical properties often correlate with stem cell differentiation². The inventors tested whether cells adhering to a non-viscous liquid displaying no bulk mechanical strength would alter stem cell fate. In FAD medium and in non-differentiating medium (KSFM), primary keratinocytes cultured on oils did not express the cornified envelop marker involucrin (below 15%, FIGS. 4A and B). In contrast, cells cultured on TPS or on oil functionalised with the protein resistant PLL-PEG remained rounded and differentiated to high levels (above 60%). This suggests that nanoscale protein interfaces are sufficient to allow cell spreading and that the lack of bulk mechanical strength did not affect keratinocyte differentiation significantly.

The lack of differentiation of keratinocytes and the high cell proliferation observed at the oil interface have direct implications for stem cell expansion for tissue engineering. Oil emulsions offer interesting features for cell culture in 3D bioreactors, whereas flat oil interfaces are attractive for the generation of cell sheets (FIG. 4C). In both cases, the absence of solid substrates allows bypassing the need for enzymatic digestion to release the cultured cells or cell sheets. To demonstrate the feasibility of such applications, the inventors cultured primary keratinocytes on oil emulsions. The inventors found that after 7 days of culture, cells had proliferated to cover the surface of droplets (FIG. 4D). Hence stem cell technologies may benefit from such liquid-liquid systems through the development of bioreactors allowing the scale up and automation of cell expansion.

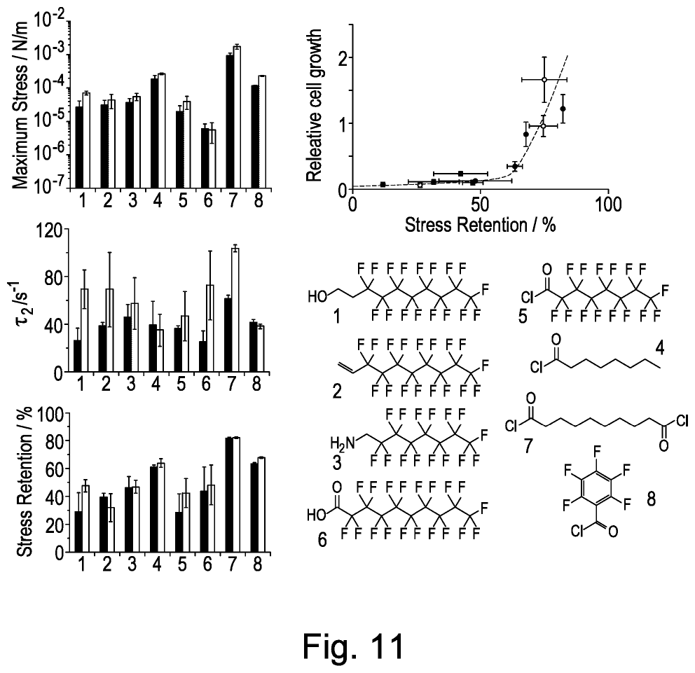

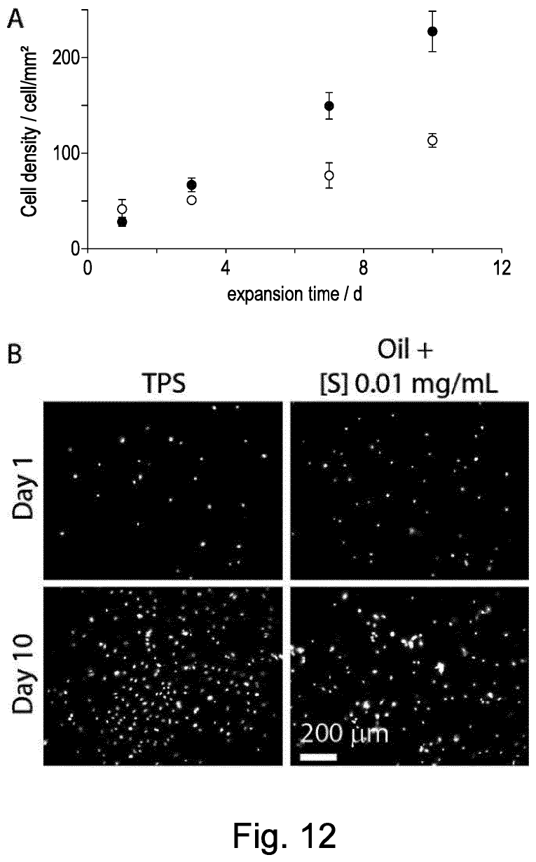

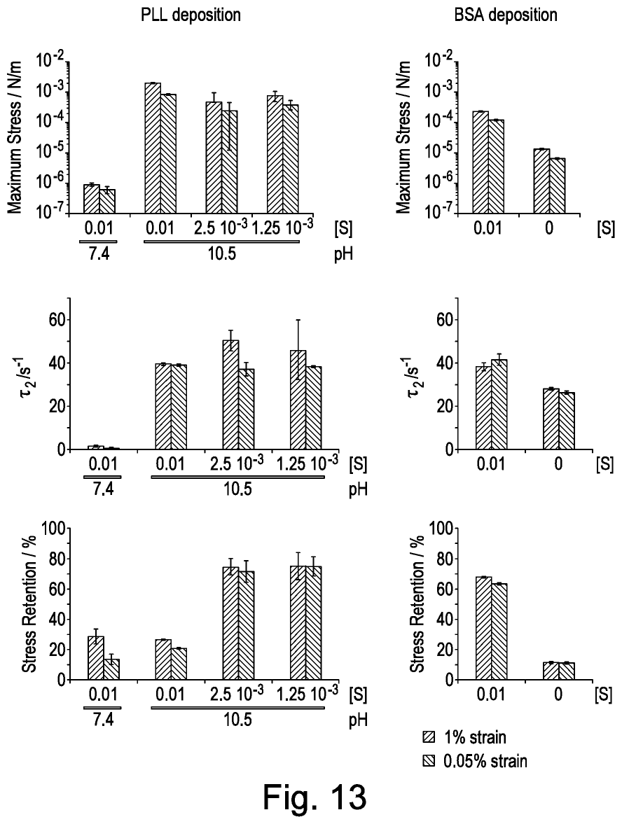

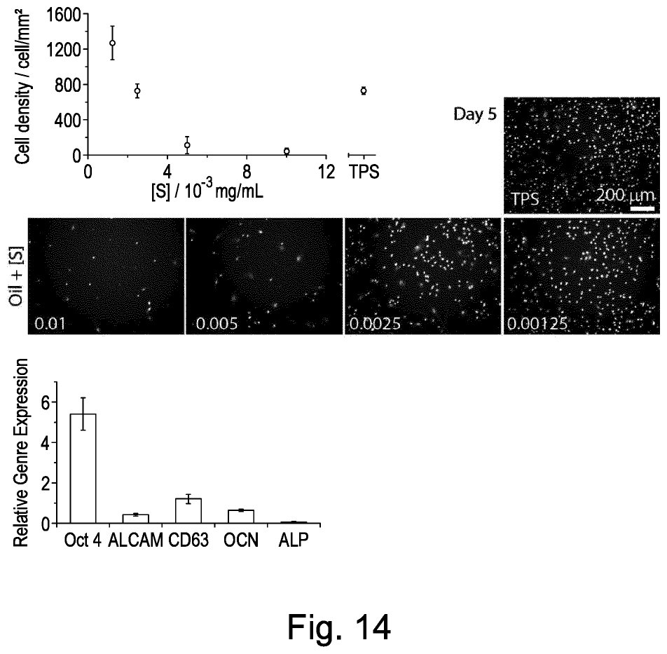

Cells were seeded at high density onto flat oil interfaces in order to study the formation of cell sheets and the resulting structures were examined via epifluorescence and confocal microscopy after transfer to a solid substrate (FIG. 4E and FIG. 11). HaCaTs generated stable sheets on fluorinated oil, displaying densities comparable to those of sheets formed on glass. F-Actin localised at the cell boundaries, indicating the formation of stable cell-cell junctions maintaining the sheet integrity and contributing to the generation of tension^{22,23}. In addition, the inventors found that actin stress fibers assembled at the basal plane of sheets, both on glass and oil, and that these structures were terminated with vinculin-rich focal adhesions (FIG. 4E and FIG. 11A). Therefore, cell sheets generated at oil interfaces displayed hallmarks of mechanical interactions with their underlying substrate. When keratinocytes were seeded instead, they formed cell multi-layers on glass substrates, with the apical layer often exhibiting increased expression of involucrin. However these sheets appeared disrupted on oil, with a large area in the centre of the sample devoid of any cell (FIG. 4E). The inventors made the hypothesis that contractile forces were responsible for the failure of sheets and repeated these experiments in medium containing the ROCK inhibitor Y27632 (FIG. 4E and FIG. 11B), which was found to allow prolonged keratinocyte expansion²⁴. Cell sheets generated in such conditions preserved their integrity, although less densely packed than sheets generated in normal FAD medium on glass. Involucrin expressing cells were also found in the apical cell layer (FIG. 11B). Hence inhibition of acto-myosin contractility prevents strong forces exerted by cell sheets from disrupting nanoscale protein assemblies at oil interfaces. This observation highlights that mechanical and physical properties of the microenvironment are not sensed at the same scale at the cellular and tissue level.

Cell adhesion to the ECM is an important process regulating the phenotype and function of many stem cells². However, from an engineering point of view the requirement for hard, rigid substrates with strong bulk mechanical properties can be an important drawback. This is the case for the scale up of cell expansion systems and the fabrication of cell sheets. Hard rigid substrates also require enzymatic digestion for cell recovery, which can be harmful or induce changes in cell phenotype (harsh trypsin treatment decreases the colony forming efficiency of keratinocytes²⁵). The use of liquid substrates directly addresses these issues and may find further application in other biotechnological platforms such a microdroplets platforms, which have been restricted by the requirements of cell adhesion²⁶. In addition, the design of biomaterials and implants should benefit from the concept that cell adhesion properties can be engineered at the interface, independently of other bulk properties that may be required to confer flexibility or structure.

### Example 7

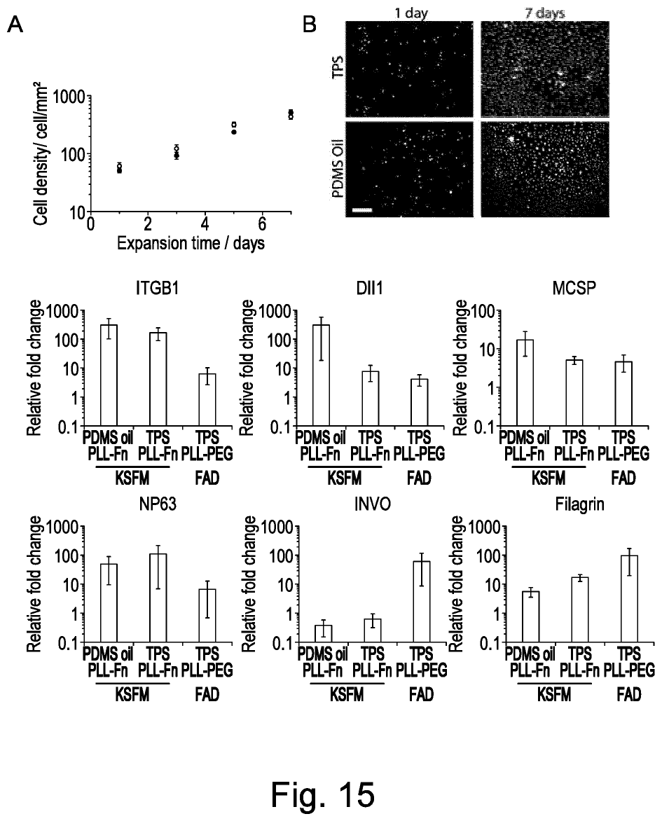

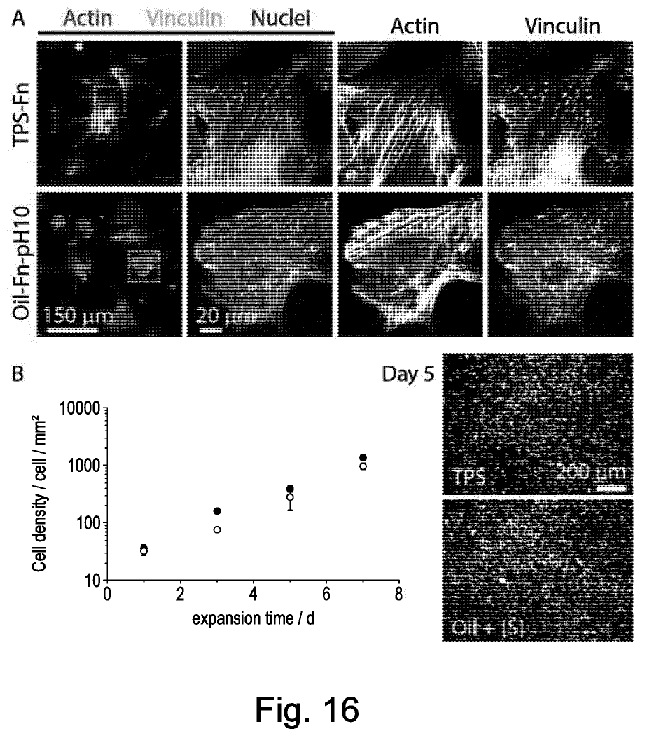

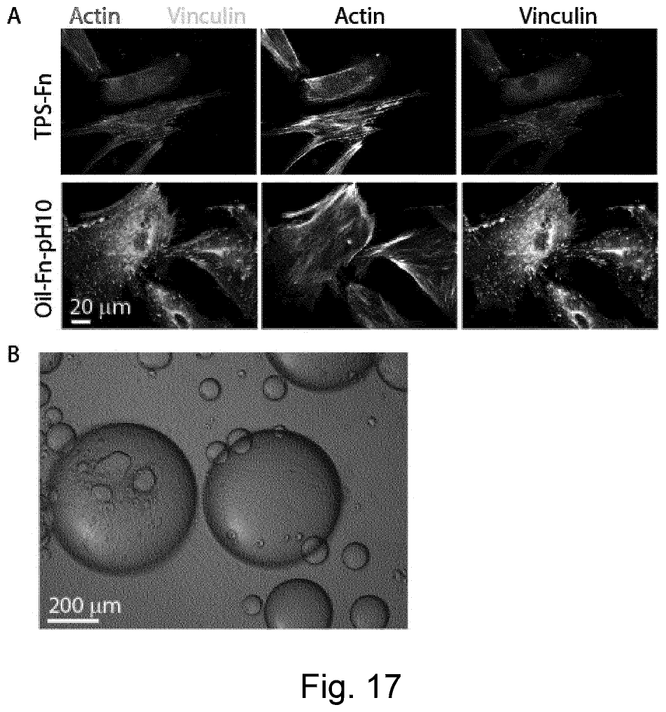

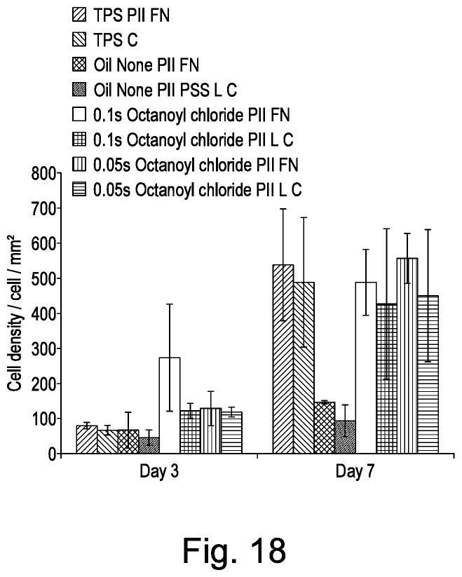

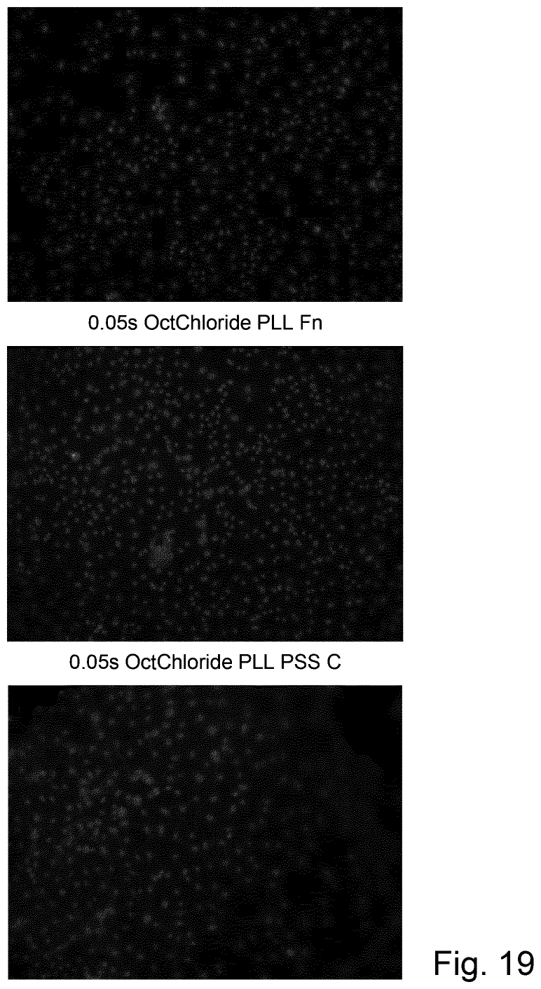

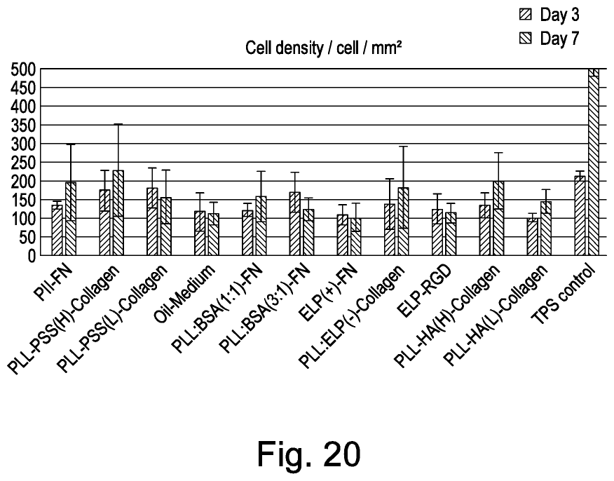

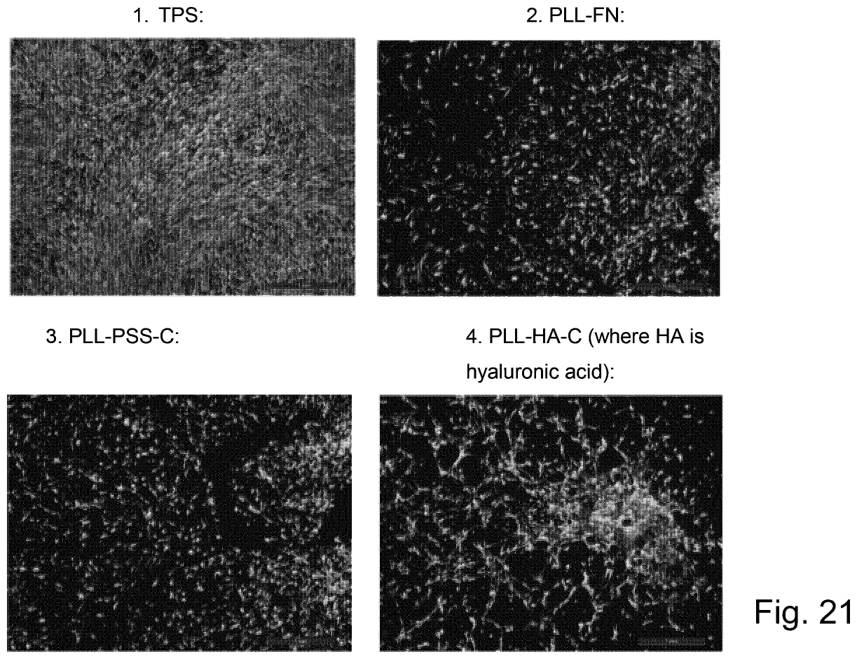

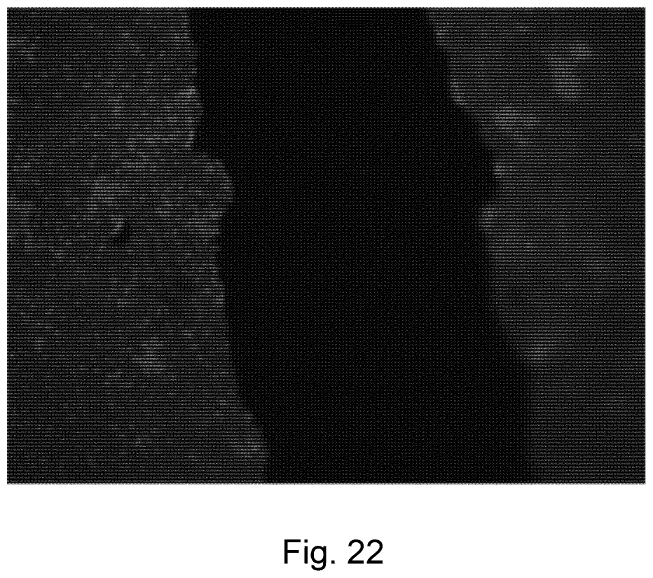

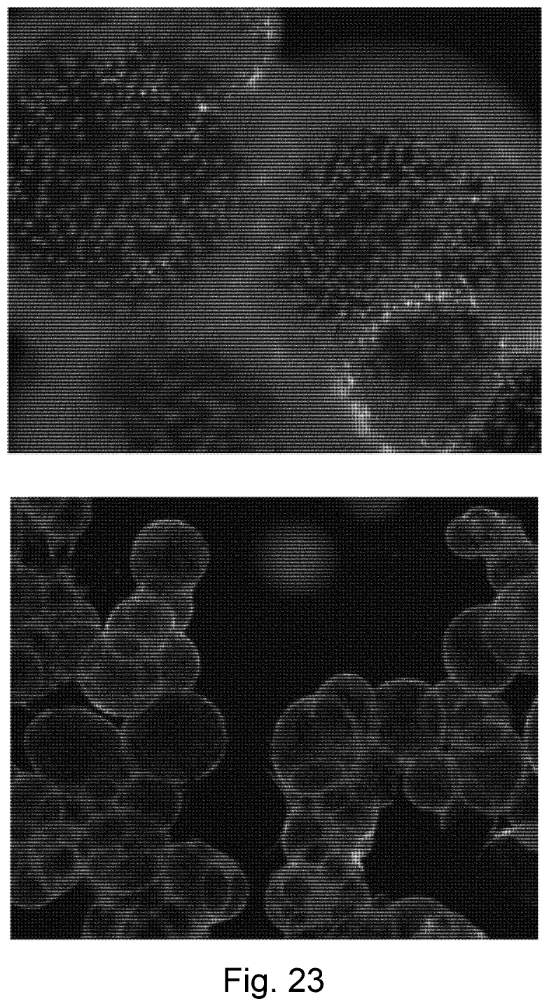

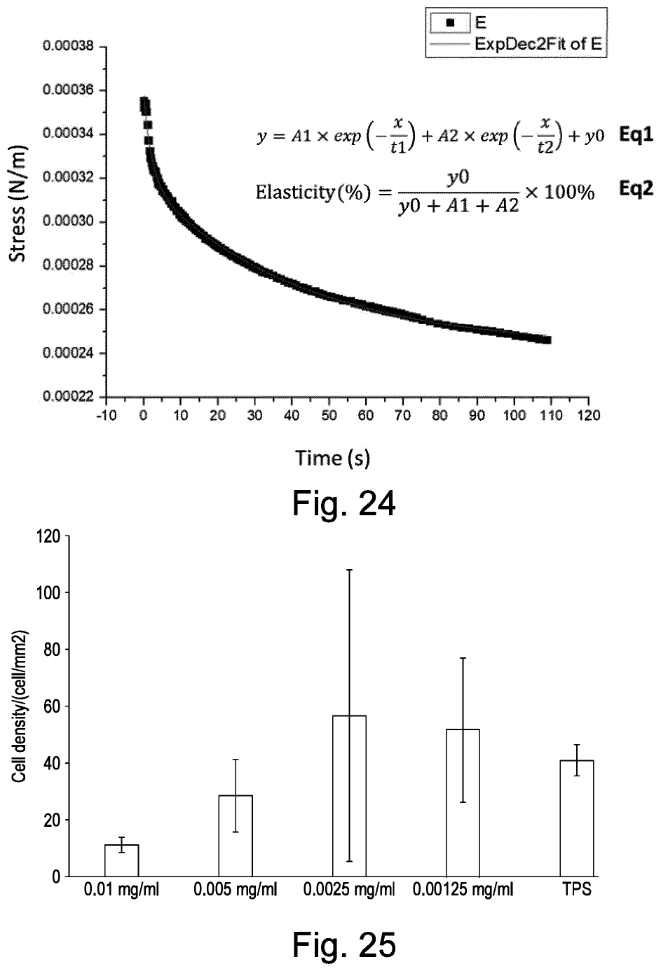

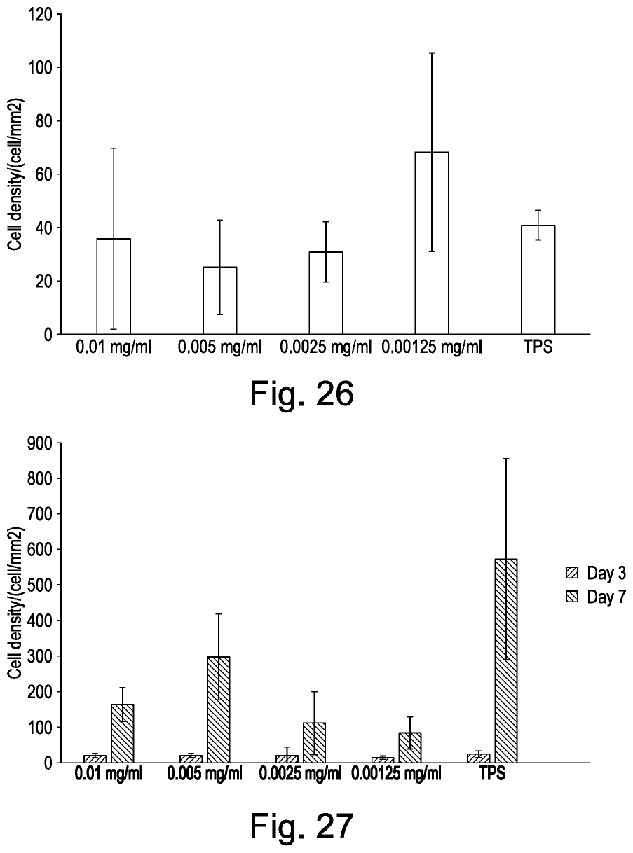

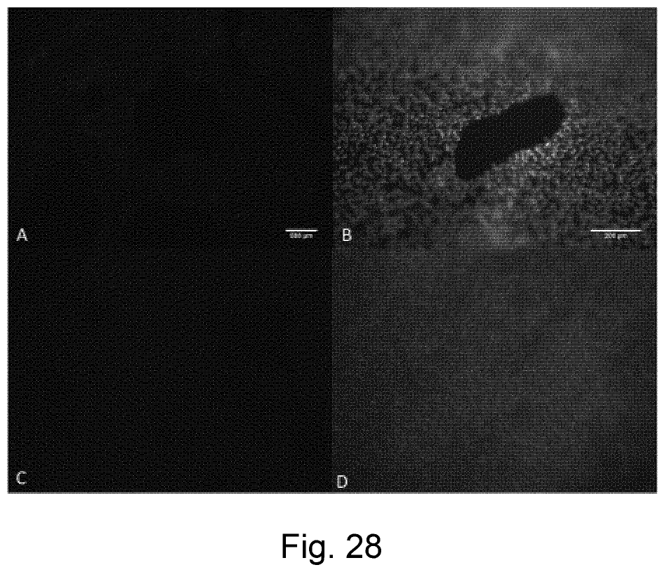

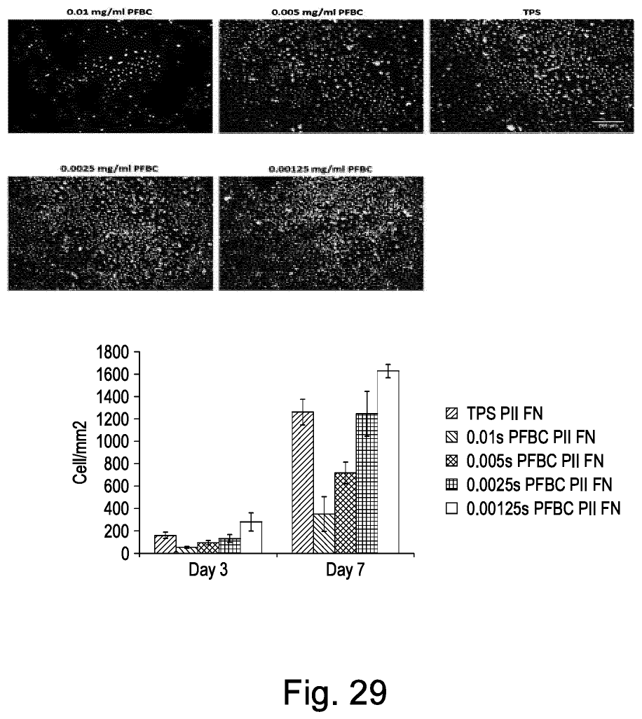

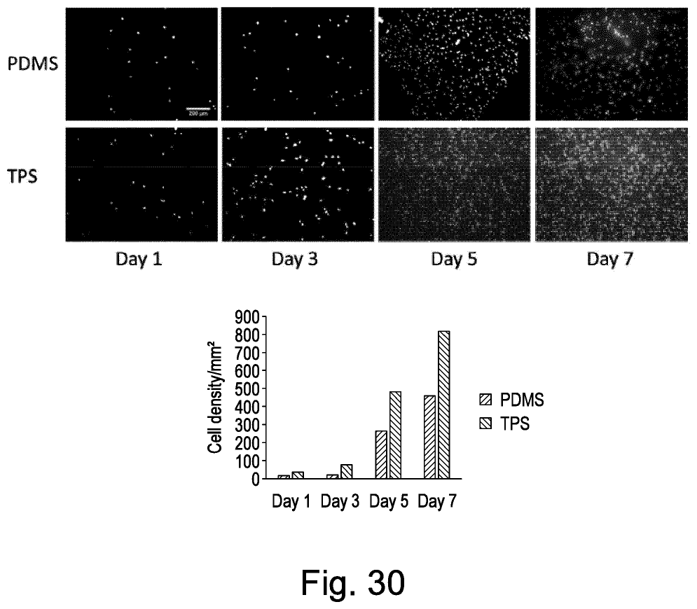

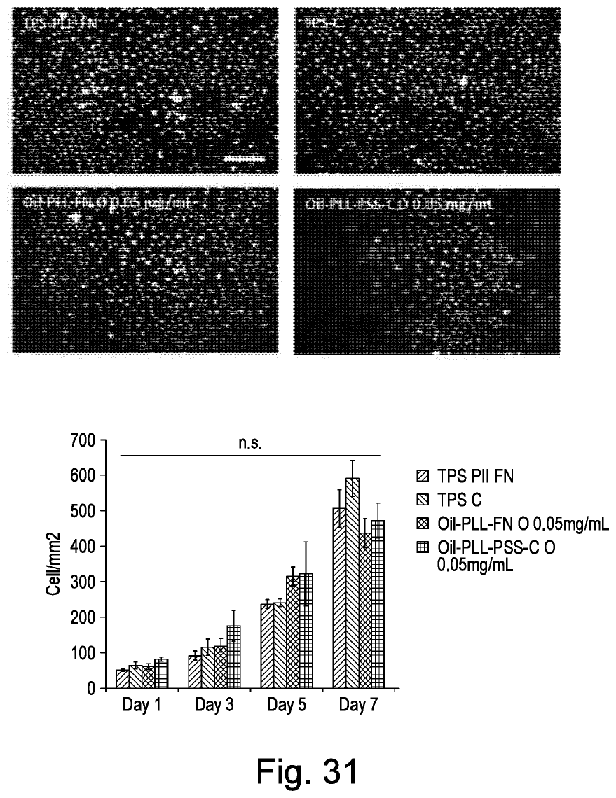

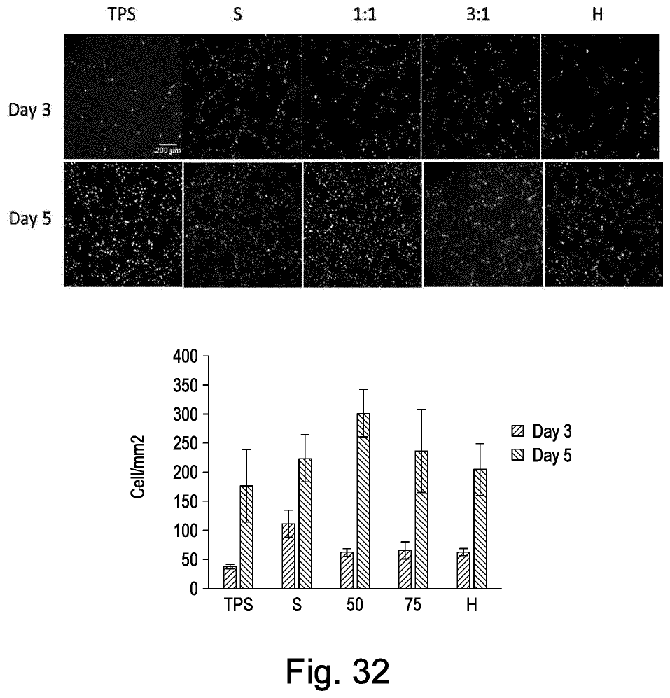

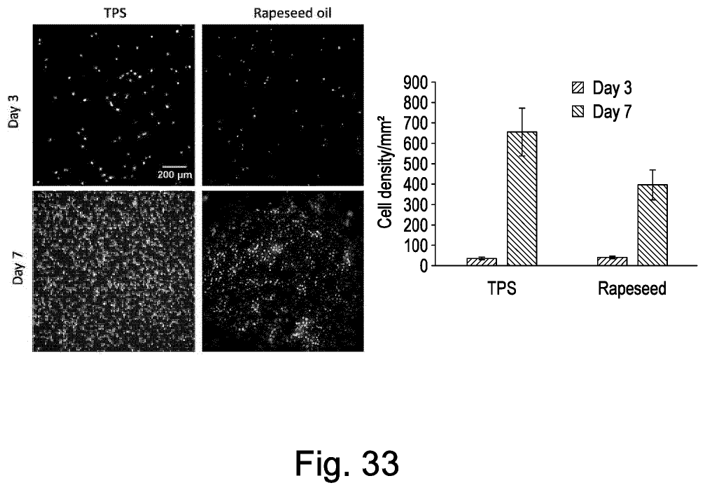

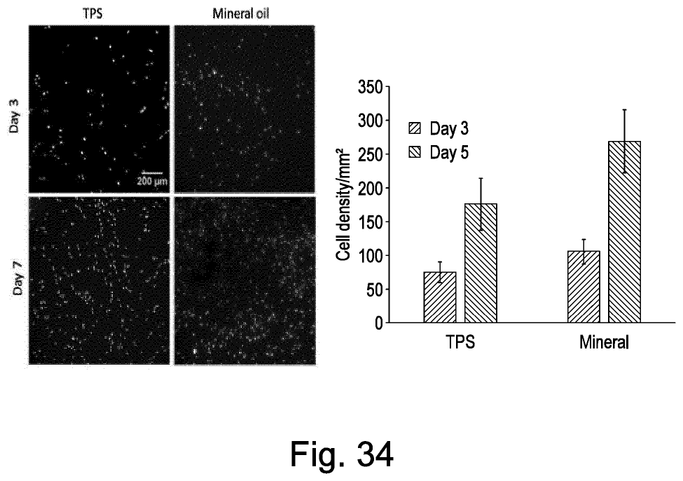

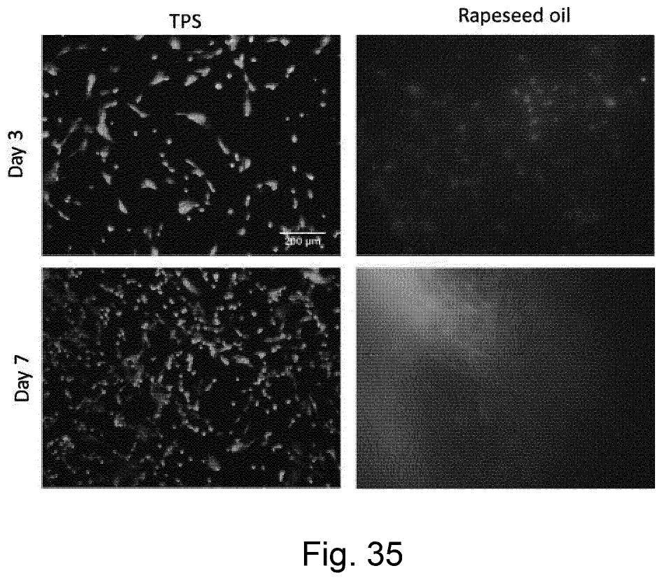

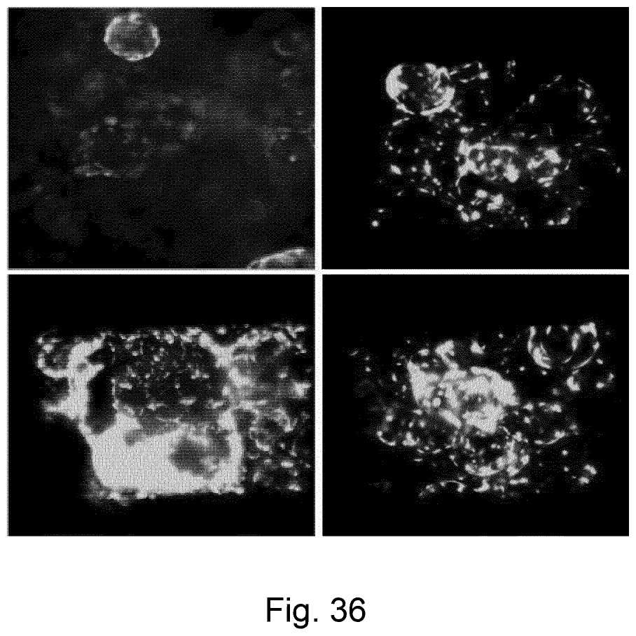

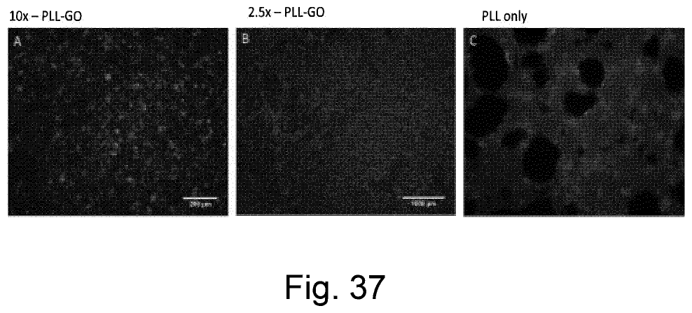

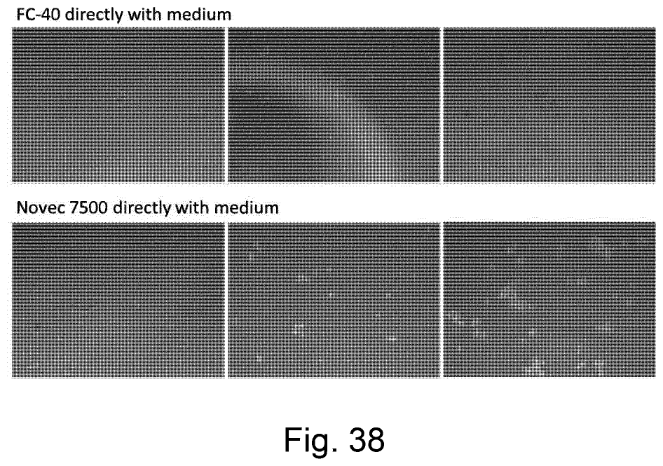

To establish the versatility of the system proposed to culture cells on liquid carriers, the inventors examined the formation of protein nanosheets on non-fluorinated oils such as silicone (PDMS) oils. To ensure compatibility of the surfactant and protein layers with the oil carriers, non-fluorinated surfactants such as octanoyl chloride, heptadecanoyl chloride and sebacoyl chloride were introduced instead of fluorinated surfactants such as PFBC. In addition to forming relatively strong protein nanosheets, it was found that these surfactants, combined with PLL allowed the stabilization of emulsions with oils such as silicone oils, mineral oil and rapeseed oil (therefore displaying a wide range of chemistries). After seeding MSCs and HPKs at low densities (typically 13,000 cells per cm²), their proliferation and viability was investigated via fluorescence microscopy (DAPI and calcein, FIGS. 15, 18, 19, 30-36) and qPCR (FIG. 15). Rates of expansion comparable to those observed on tissue culture polystyrene were observed and our results demonstrated the retention of stem cell markers (qPCR). Hence these results demonstrate that stem cell culture and expansion with retention of stem cell markers can be promoted at the surface of non-fluorinated oils with a wide range of chemistries.

### Example 8

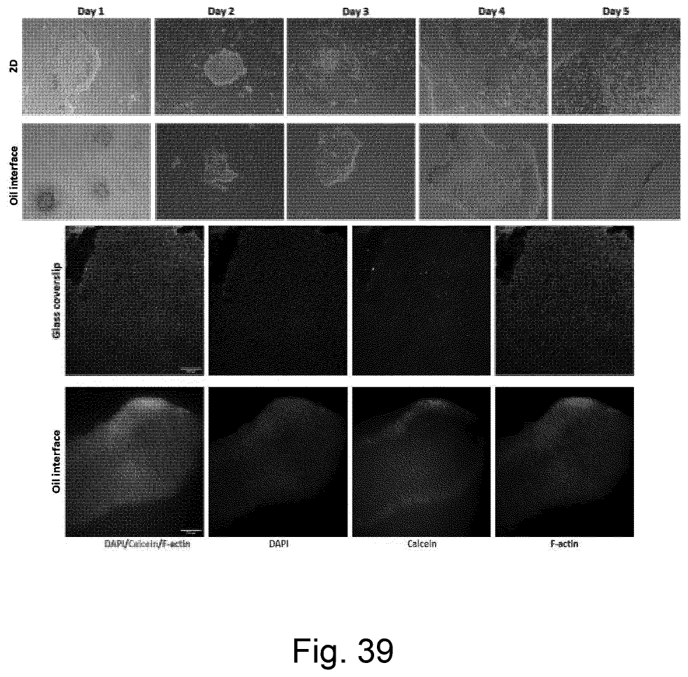

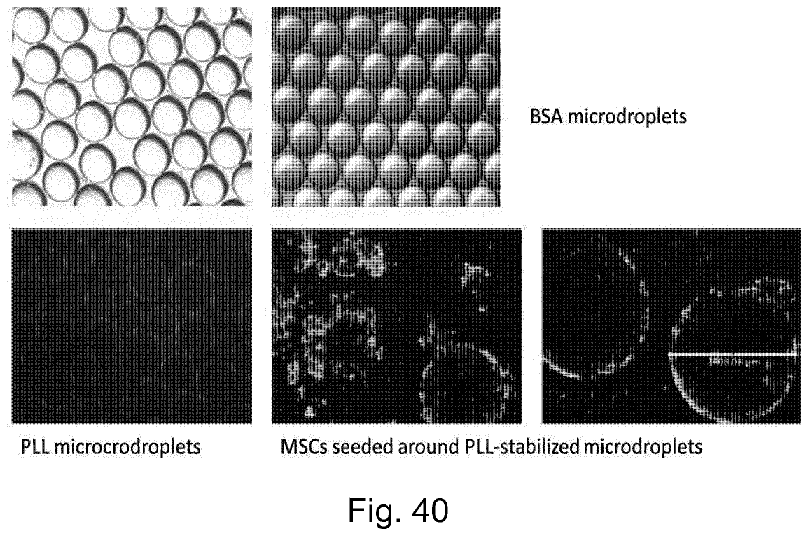

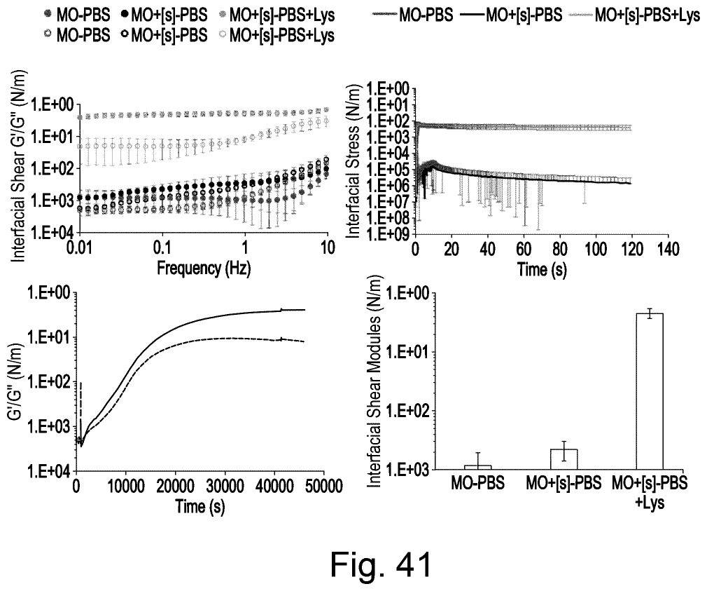

To further establish the versatility of this system of culture of stem cells at liquid interfaces, the inventors culture induced pluripotent stem cells (iPSCs) at the surface of Novec 7500 oil, supplemented with PFBC (0.00125 mg/mL) to promote the assembly of PLL nanosheets. To promote the expansion of iPSCs at such interfaces, vitronectin was deposited on PLL nanosheets instead of fibronectin (10 μg/mL), further demonstrating that a range of ECM proteins can be assembled onto nanosheets to promote cell and stem cell expansion on liquid carriers. The iPSC colonies formed in such conditions were similar in size to those formed on tissue culture plastic, although a little slower (FIG. 39). However, their morphology was more rounded and less scattered on oil interfaces than on plastic, suggesting a reduction in stem cell differentiation during long term culture. Therefore these results, combined to other results obtained for the culture of primary keratinocytes and mesenchymal stem cells, demonstrate that a broad range of stem cells can be cultured at the surface of liquid carriers.

### METHODS

Generation of liquid-liquid interfaces for cell culture. 24 well plates were plasma oxidized using a plasma coater (Diener, 100% intensity) for 10 minutes. 500 tL ethanol (VWR chemicals), 10 tL trimethylamine (Sigma-Aldrich) and 10 tL of the desired silane (triethoxy(octyl)silane (Sigma-Aldrich) to prepare interfaces with liquid PDMS or trichloro (1H, 1H, 2H, 2H-perfluorooctyl) silane (Sigma-Aldrich) for the fluorinated oil) were added into each well. Ethanol was added in between wells to slow down evaporation and parafilm was used to seal the well plate cover. After incubating for 24 h, the wells were washed in a sterile environment with ethanol (twice) and ddH2O (three times). 500 tL fluorinated oil (Novec 7500, ACOTA) with fluorinated surfactant (2,3,4,5,6-pentafluorobenzoyl chloride, Sigma-Aldrich) at final concentrations of 10, 1, 0.1, 0.01, 0.005, 0.001 and 0 mg/mL was added in the fluorophilic (or oleophilic in the case of PDMS interfaces) 24 well plate to form the bottom liquid layer. Conditioning of the surface was carried out with bovine serum albumin (BSA, 1 mg/mL, Sigma-Aldrich), collagen (type I, 20 μg/mL, Corning) or culture medium (supplemented with foetal bovine serum, 10%, Labtech) solutions and incubated for 20 min. To wash out these protein solutions, dilutions with sterile PBS (Sigma-Aldrich, 3 times) was carried out, followed by dilution with growth medium.

Generation of PDMS droplet substrates for cell culture. Thin glass slides (25×60 mm, VWR) were plasma oxidized for 10 minutes and placed into a staining jar. 100 tL triethoxy(octyl)silane (Sigma-Aldrich), 100 tL triethylamine (Sigma-Aldrich) and toluene (Sigma-Aldrich, 50 mL) were added to the jar. The jar was covered and sealed with parafilm and left in a fumehood overnight. The resulting ydrophobic thin glass slides were cut into chips (1×1 cm) and placed into a 24 well plate. After sterilisation with 70% ethanol, the wells were washed (twice) and filled with 2 mL PBS (Sigma-Aldrich). 100 tL of liquid PDMS droplets (with viscosities of 10, 50, 1000, 3500 (Sigma-Aldrich) and 5000 cst, all from ABCR unless specified; Sylgard 184 was purchased from Ellsworth) were added on top of the glass slide, resulting in a PDMS droplet that covered approximately 75% of the surface. The PBS contained within the wells was diluted with growth medium twice.

Generation of fluorinated oil droplet substrates for cell culture. Thin glass slides (25×60 mm, VWR) were plasma oxidized for 10 minutes and placed into a staining jar. Toluene (1 mL, Sigma-Aldrich) and 30 μL trichloro (1H, 1H, 2H, 2H-perfluorooctyl) silane (Sigma-Aldrich) were added in a glass vial. The staining jar containing the glass slides and the glass vial with the silane solution were placed into a desiccator under vacuum for 5 min and then left under reduced atmosphere but sealed overnight. The fluorinated glass slides were cut into chips (1×1 cm) and placed into a 24 well plate. After sterilisation with 70% ethanol, the wells were washed (twice) and then filled with 2 mL PBS (pH 7.4, except when specified, for the adsorption of poly(L-lysine) at pH 10.5). 100 tL droplets of fluorinated oil (Novec 7500) with fluorinated surfactant (2,3,4,5,6-Pentafluorobenzoyl chloride) at specified concentrations (see specific experiments, but often 0.01 mg/mL) were deposited on top of the glass slide and formed a fluorinated oil droplet spreading over the entire substrate. 30 tL oil was removed by micropipette to form a flatter and more stable oil droplet. For the deposition of poly(L-lysine) (PLL), a 20 μL PLL solution (10 mg/mL) was added to PBS, to make a final concentration of 100 μg/mL, and incubated for 1 h. The protein solution was then diluted with PBS (pH 7.4) 6 times. For fibronectin adsorption, 20 μL fibronectin solution (1 mg/mL) was pipetted into the well (after PLL coating), making a final concentration of 10 μg/mL, and incubated for 1 h. The protein solution was diluted with PBS (PH 7.4) 4 times and then with growth medium twice. For the deposition of BSA, a 10 μL BSA solution (100 mg/mL) was added into PBS, to make a final concentration of 1 mg/mL, and incubated for 1 h. For the deposition of poly(L-lysine)-graft-poly(ethylene glycol) (PLL-PEG, Surface Solutions), oil droplets were exposed to solutions of PLL-PEG (100 μg/mL) for 1 h at pH 10.5.

Generation of emulsions for characterisation and cell culture. 1 mL fluorinated oil (Novec 7500, ACOTA) containing the fluorinated surfactant 2,3,4,5,6-Pentafluorobenzoyl chloride at final concentrations of 10, 1, 0.1, 0.01, 0.005, 0.001 and 0 mg/mL and 2 mL BSA (1 mg/ml solution in PBS) were added into a 15 ml centrifuge tube. The tube was vigorously shaken manually to mix and generate the emulsion and subsequently left to incubate at room temperature for 1 h. The top liquid phase, above the settled emulsion was aspirated and replaced with PBS 6 times and deionised water twice. Droplets of emulsion were then transferred to silicon substrates (silicon wafer, Pi-Kem Ltd.) and left to dry in air for SEM, XPS and AFM analysis (see below). Samples analysed by XPS and AFM were further washed with ethanol before drying.

For cell culture, fibronectin was deposited at the surface of oil droplets after PLL adsorption. 1 mL fluorinated oil (Novec 7500) with fluorinated surfactant (2,3,4,5,6-Pentafluorobenzoyl chloride 0.01 mg/mL) and 2 mL of PLL solution (200 μg/mL) in pH10.5 PBS were added in a 15 mL centrifuge tube. The tube was vigorously shaken to mix and form the emulsion and subsequently left to incubate at room temperature for 1 h. The top liquid phase above the settled emulsion was aspirated and replaced with PBS 6 times. 20 μL of human plasma fibronectin (1 mg/mL) was added (final concentration of 10 μg/mL) and incubated at room temperature for 1 h. The top liquid phase above the emulsion was aspirated and replace with PBS 6 times. For cell seeding, 2 mL of growth medium was added in a 24 well plate and 500 μL of the emulsion were transferred to the well.

Keratinocyte culture and seeding. Primary human epidermal keratinocytes isolated from neonatal foreskin were cultured on collagen I (type 1, Corning, 20 μg/mL in PBS for 20 min) treated T75 flask in keratinocyte serum free medium KSFM (Thermofisher Scientific) supplemented with Bovine Pituitary Extract (BPE) and EGF (Human Recombinant). Keratinocytes were harvested with trypsin (0.25%, Thermofisher Scientific) and versene solutions (Thermofisher Scientific, 0.2 g/L EDTA Na4 in Phosphate Buffered Saline) in a ratio of 1/9, centrifuged, counted and resuspended in KSFM at the desired density before seeding onto substrates at a density of 25,000 cells per well (13,000 cells per cm²). Cells were left to adhere for 24 h in an incubator (37° C. and 5% CO2). For the generation of keratinocytes cell sheets, 200,000 cells were seeded on fluorinated oil droplets generated on large fluorinated glass slides (2×2 cm) in FAD medium (consisting of half Ham's F12 and half DMEM, Thermofisher Scientific) supplemented with 10% foetal bovine serum (FBS), 1% L-Glutamine (200 mM) (Thermofisher Scientific) and 1% Penicillin-Streptomycin (5,000 U/mL) (Thermofisher Scientific), 0.1% HCE (Thermofisher Scientific) and 0.1% insulin (Thermofisher Scientific), in a 6-well plate. When cultured in the presence of the ROCK inhibitor Y-27632 (R&D Systems), the inhibitor was added at a final concentration of 10 tM from a 10 mM DMSO stock solution for 24 h. For the cell adhesion and differentiation assays, keratinocytes were seeded at a density of 25,000 cells per well (13,000 cells per cm²) in a 24-well plate, in the relevant medium (KSFM or FAD) as stated in the figures, and left in the incubator for 24 h prior to fixation and immunostaining. For passaging, cells were reseeded in a T75 at a density of 250 k cells per flask.

HaCaT keratinocyte cell line culture and seeding. Human keratinocyte HaCaT cells were cultured in DMEM (Thermofisher Scientific) containing 10% foetal bovine serum (FBS, Labtech), 1% L-Glutamine (200 mM) and 1% Penicillin-Streptomycin (5,000 U/mL). For proliferation assays, HaCaT cells were harvested with trypsin (0.25%) and versene solutions (Thermofisher Scientific, 0.2 g/L EDTA Na4 in Phosphate Buffered Saline) in a ratio of 1/9, centrifuged, counted and resuspended in DMEM at the desired density before seeding onto substrates (conditioned as stated above, in a 24-well plate) at a density of 2,000 per well (1,000 cells per cm²). Cells were left to adhere and proliferate in an incubator (37° C. and 5% CO2) for different time points, prior to staining and imaging. For cell spreading assays HaCaT cells were harvested and seeded onto fluorinated droplets at a density of 25,000 per well (13,000 per cm²). For passaging, cells were reseeded in a T75 at a density of 250 k cells per flask.

Immunofluorescence staining and antibodies. After washing once with PBS, samples were fixed with 4% paraformaldehyde (Sigma-Aldrich) for 10 min and permeabilized with 0.2% Triton X-100 (Sigma-Aldrich) for 5 min at room temperature. Following blocking for 1 h in 10% foetal bovine serum and 0.25% gelatine (from cold water fish skin, Sigma-Aldrich), substrates were incubated with primary antibodies for 1 h at room temperature, and with Alexa Fluor 488-conjugated secondary antibody (Thermofisher Scientific) for 1 h at room temperature (1:1000). When relevant, tetramethyl rhodamine isothiocyanate phalloidin (1:500, Sigma-Aldrich) was included in the blocking solution and DAPI in the secondary antibody solution. Samples of cells adhering to oils were imaged directly without mounting. Cell sheet samples, after transfer to a solid substrate, were mounted on glass slides with Mowiol reagent (4-88, Sigma-Aldrich). The Mouse anti-vinculin (hVIN1, 1:1000) was purchased by Sigma-Aldrich. The Mouse anti-involucrin (SY7; 1:1000) was prepared by Cancer Research UK central services.

Hoechst staining and LIVE/DEAD cell viability assay. Cell proliferation was assessed via Hoechst staining. Cells were incubated in DMEM containing 5 μL Hoechst 33342 (1 mg/mL, Thermofisher Scientific) for 30 min before imaging by epifluorescence microscopy (see below). Viability of HaCaT cells on fluorinated oil interfaces was quantified by LIVE/DEAD viability/cytotoxicity assay using a kit supplied by Thermofisher Scientific. In brief, HaCaT cells were incubated in DMEM with 2 μM Calcein AM and 4 μM Ethidium homodimer for 30 min. stained cells were imaged using a Leica DM14000 fluorescence microscopy (see below). The percentage of viable cells was calculated by counting the number of green (live) cells and dividing by the total number of cells (including dead cells).

Immuno-fluorescence microscopy and data analysis. Fluorescence microscopy images were acquired with a Leica DMI4000B fluorescence microscopy (CTR4000 lamp; 63×1.25 NA, oil lens; 10×0.3 NA lens; 2.5×0.07 NA lens; DFC300FX camera). Confocal microscopy images were acquired with a Leica TCS SP2 confocal and multiphoton microscope (X-Cite 120 LED lamp; 63×1.40-0.60 NA, oil lens; 10×0.3 NA lens; DFC420C CCD camera). To determine cell densities per mm², cell counting was carried out by thresholding and watershedding nuclei images in ImageJ. In the case of cell clumps, for which this protocol did not allow the isolation of individual nuclei, cells were counted manually. To determine adhesion cell areas, images were analyzed by thresholding and watershedding fluorescence images of the cytoskeleton (phalloidin stained). The area of cell clusters was removed when analysing results.

For confocal imaging stacks of 16 sections were scanned, with an image averaging of 2 and a line averaging of 4. 3D reconstruction and volume rendering of the stacks were performed with the appropriate plugins of Imaris ×64. Statistical analysis was carried out using Origin 8 through one-way ANOVA with Tukey test for posthoc analysis. Significance was determined by * P<0.05, ** P<0.01, *** P<0.001 and n.s., non-significant. A full summary of statistical analysis is provided below as a separate supporting file.

Scanning electron microscopy. The dried protein-coated droplets deposited on silicon substrates were coated with gold for 30 s before imaged using an FEI Inspect F scanning electron microscopy operated at 10 kV. A spot size of 3.5 and an aperture of 30 mm were used. Experiments were repeated twice and five areas were analysed at different magnifications (140×, 1200×, 8000× and 80000×).

SICM imaging of cells on oil droplet. Fluorinated glass slides (see above) were cut into 2×1 cm rectangle and placed in a 50 cm² petri dish. After sterilisation with 70% ethanol, the wells were washed (twice) and then filled with 5 mL PBS (pH 7.4, except when specified, for the adsorption of poly(L-lysine) at pH 10.5). 20 μL droplets of fluorinated oil (Novec 7500) with fluorinated surfactant (2,3,4,5,6-Pentafluorobenzoyl chloride) at 0.01 mg/mL were deposited on top of the glass slide and formed a fluorinated oil droplet spreading over the entire substrate. 10 μL oil was removed by micropipette to form a flatter and more stable oil droplet. For the deposition of poly(L-lysine) (PLL), a 50 μL PLL solution (10 mg/mL) was added to PBS, to make a final concentration of 100 μg/mL, and incubated for 1 h. The protein solution was then diluted with PBS (pH 7.4) 6 times. For fibronectin adsorption, 50 μL fibronectin solution (1 mg/mL) was pipetted into the well (after PLL coating), making a final concentration of 10 μg/mL, and incubated for 1 h. The protein solution was diluted with PBS (PH 7.4) 4 times and then with KSFM medium (Thermofisher Scientific) twice. Then 100,000 cells were seeded on each petri dish for 24 h. After dilution three times with PBS, samples were fixed with 4% paraformaldehyde (Sigma-Aldrich) for 10 min and diluted 6 times with PBS. The topographical images of cells were obtained using a custom built SICM setup operating in the “hopping mode” at setpoint of 0.3% as described previously. Glass nanopipettes with estimated inner diameter of 74 nm pulled from borosilicate glass capillaries with 0.5 mm inner and 1.0 mm outer diameter were used in all SICM experiments.

Atomic force microscopy (AFM). The dried protein-coated droplets deposited on a silicon substrate were directly imaged by AFM without further treatment. In order to assess the thickness of the protein layers, the samples were gently scratched in different point with the tip of metal tweezers (Dumont). For the imaging, an AFM (NT-MDT NTEGRA) was used in semi contact mode topography. The surface of the substrate was visualised via an optical microscope to identify the localisation of the scratches. The scans were conducted at a frequency of 1.01 Hz. The areas scanned were of 50 by 50 μm. The probes used were for non-contact mode from NT-MDT (resonant frequency between 87-230 kHz and force constant 1.45-15.1 N/m). Pictures were corrected via the software subtracting a 1^{st} order curve. The profiles across the scratch were then analysed and the differences in height were measured via the software, affording a direct measurement of the thickness of the dried protein layer (after correcting by a factor of 2 as the drying of a single droplet results in the deposition of two protein layers). Experiments were repeated twice and five areas were analysed for each condition, taking between 5 and 10 measurements for each scan.

X-ray photoelectron spectroscopy. XPS was carried out using a Kratos Axis Ultra DLD electron spectrometer with a monochromated Al Kα source (1486.6 eV) operated at 150 W. A pass energy of 160 eV and a step size of 1 eV were used for survey spectra. For high energy resolution spectra of regions, a pass energy of 20 eV and a step size of 0.1 eV were used. The spectrometer charge neutralising system was used to compensate sample charging and the binding scale was referenced to the aliphatic component of C 1s spectra at 285.0 eV. The concentrations obtained (error less than ±10%) are reported as the percentage of that particular atom species (atomic %) at the surface of the sample (<10 nm analysis depth) without any correction. The analysis area (0.3 [symbol] 0.7 mm²), the angle of incidence and the beam intensity were kept constant for all measurements. To determine the functionalisation level of BSA macromolecules with PFBC surfactant, atomic % reported in the literature were used (62.6% for C 1s, 14.4% for N 1s and 23.0 for O 1s, see Adler, M., Unger, M. & Lee, G. Pharm. Res. 17, 863-870, 2000) and a molecular weight of 66 kDa was used in the calculations to determine a standard curve (and equation) predicting the evolution of the F 1s atomic % as a function of the number of PFBC surfactant tethered. For the functionalisation of PLL, this calculation was directly based on the molar mass of lysine repeat units.

Attenuated Total Reflectance—Fourier Transformed Infrared (ATR-FTIR) Spectroscopy. All ATR-FTIR measurements were performed on a Brucker Tensor 27 infrared spectrometer equipped with an MCT detector, cooled with liquid N2. 2 ml fluorinated oil (Novec 7500, ACOTA) with fluorinated surfactant (2,3,4,5,6-Pentafluorobenzoyl chloride) at final concentrations of 10 mg/mL and 4 ml BSA 1 mg/mL solution were added into a glass vial. The vial was vigorously shaken manually to mix and form the emulsion and subsequently left to incubate at room temperature for 1 h. To wash the emulsion, the top liquid phase, above the settled emulsion was aspirated and replaced with PBS 6 times and deionised water twice. The glass vials was then left overnight under vacuum resulting in a dry white residue. One sample of this dry material was washed further with ethanol to remove any unbound surfactant, whilst one sample was directly analysed by FTIR spectroscopy. The dried residues, 2,3,4,5,6-Pentafluorobenzoyl chloride, and BSA powder were characterised by FTIR spectroscopy.

Interfacial rheology. Rheological measurements were carried out on a hybrid rheometer (DHR-3) from TA Instruments fitted with a double wall ring (DWR) geometry and a Delrin trough with a circular channel. The double wall ring used for this geometry has a radius of 34.5 mm and the thickness of the Platinum-Iridium wire is 1 mm. The diamond-shaped cross-section of the geometry's ring provides the capability to pin directly onto the interface between two liquids and measure the interface properties without complicated sub-phase correction. 19 mL of the fluorinated oil pre-mixed with surfactant were placed in the Delrin trough and the ring was lowered, ensuring contact with the surface, via an axial force procedure. The measuring position was set 500 μm lower than the contact point of the ring with the oil-phase surface. Thereafter, 15 ml of the PBS buffer were carefully syringed on top of the oil phase. Time sweeps were performed at a constant frequency of 0.1 Hz and a temperature of 25° C., with a displacement of 1.0 10⁻³ rad to follow the formation of the protein layers at the interface. The concentration of BSA used for all rheology experiments was 1 mg/mL (with respect to aqueous phase volume). Before and after each time sweep, frequency sweeps (with a constant displacement of 1.0 10⁻³ rad) were conducted to examine the frequency-dependant characteristics of the interface whilst amplitude sweeps (with constant frequencies of 0.1 Hz) were carried out to ensure that the chosen displacement was within the linear viscoelastic region.

Rheology on cured PDMS. Sylgard 184 PDMS samples were prepared at a base/cross linker ratio of 100/1. The base and corsslinker were mixed and degassed until no bubbles were present under vacuum. The sample was then tested in a TA Discovery HR3 Rheometer. The PDMS was initially cured in situ at 60 degrees Celsius for 3 hrs under oscillating time sweep at a frequency of 1 Hz and oscillating amplitude of 1 10⁻⁴ rad. Once the sample had cured it was cooled to room temperature (25° C.) and left to settle for 300 s before a series of stress relaxation tests were performed. All relaxation tests were 300 s long with a strain rate of 1%/s and the samples were strained to 0.01%, 0.1%, 1% and 10%. After the stress relaxation tests a frequency sweep was performed at a displacement of 1 10⁴ rad from 0.1 to 100 Hz on a logarithmic scale getting 10 points per decade. This was followed by a stress sweep performed at 1 Hz, from 0.1 to 100 Pa on a logarithmic scale with 10 points per decade.

(PLL GO)-(PLL GO)-(PLL GO)-PLL-Fibronectin composites deposition on liquid-liquid interface. Thin glass slides (25×60 mm, VWR) were plasma oxidized for 10 minutes and placed into a staining jar. Toluene (1 mL, Sigma-Aldrich) and 30 μL trichloro (1H, 1H, 2H, 2H-perfluorooctyl) silane (Sigma-Aldrich) were added in a glass vial. The staining jar containing the glass slides and the glass vial with the silane solution were placed into a desiccator under vacuum for 5 min and then left under reduced atmosphere but sealed overnight. The fluorinated glass slides were cut into chips (1×1 cm) and placed into a 24 well plate. After sterilisation with 70% ethanol, the wells were washed (twice) and then filled with 2 mL pH 10.5 PBS. 100 μL droplets of fluorinated oil (Novec 7500) with fluorinated surfactant (2,3,4,5,6-Pentafluorobenzoyl chloride 10 mg/mL) were deposited on top of the glass slide and formed a fluorinated oil droplet spreading over the entire substrate. 30 μL oil was removed by micropipette aspiration to form a flatter and more stable oil droplet.

Subsequently, a labelled PLL solution (2 μL PLL-Alexa Fluor™ 594 at 10 mg/mL, mixed with 18 μL of PLL solution at 10 mg/mL) was added to PBS to make a final PLL concentration of 100 μg/mL, and the resulting interfaces were incubated for 1 h. The protein solution was then diluted with PBS (pH 7.4) 6 times. Graphene oxide was next deposited on the resulting interfaces: a graphene oxide solution (GO, 200 μg/mL) was pipetted into the well (after PLL coating), making a final concentration of 100 μg/mL, and incubated for 0.5 h. The GO (graphene oxide) solution was then diluted with PBS (pH 10.5) 6 times. The PLL-GO adsorption process was repeated twice more, to afford the (PLL-GO)-(PLL-GO)-(PLL-GO) composites adsorbed on the corresponding interface. Finally, a labelled PLL solution was incubated for 0.5 h (final concentration 100 μg/mL) and diluted with PBS (PH 7.4) 6 times; followed by a fibronectin adsorption, 50 μL fibronectin solution (1 mg/mL) was pipetted into the well, making a final concentration of 10 μg/mL, and incubated for 1 h. Cells were subsequently seeded and cultured on the resulting interfaces as for other liquid interfaces.

Generation of PDMS emulsions for MSCs culture. 1 mL PDMS (for example with a viscosity of 10 cSt) containing sebacoyl/heptadecanoyl chloride mixed at 1:1 ratio at 0.01 mg/ml concentration and 2 mL of PLL solution (200 μg/mL) in pH10.5 PBS were added in a glass vial. The vial was vigorously shaken to form the emulsion and subsequently left to incubate at room temperature for 1 h. The bottom liquid phase below the settled emulsion was aspirated and replaced with PBS 4 times. 20 μL of human plasma fibronectin (1 mg/mL) was added (final concentration of 10 μg/mL) and incubated at room temperature for 1 h. The bottom liquid phase below the emulsion was aspirated and replace with PBS 3 times. For cell seeding, 2 mL of growth medium was added in a 24 well plate and 500 μL of the emulsion were transferred to the well, 20 k cells were seeded.

Generation of liquid-liquid (FC-40) interfaces for cell culture. 24 well plates were plasma oxidized using a plasma coater (Diener, 100% intensity) for 10 minutes. 500 mL ethanol (VWR chemicals), 10 mL trimethylamine (Sigma-Aldrich) and 10 mL of the desired silane (triethoxy(octyl)silane (Sigma-Aldrich) to prepare interfaces with liquid PDMS, or trichloro (1H, 1H, 2H, 2H-perfluorooctyl) silane (Sigma-Aldrich) for the fluorinated oil) were added into each well. Ethanol was added in between wells to slow down evaporation and parafilm was used to seal the well plate lid. After incubating for 24 h, the wells were washed in a sterile environment with ethanol (twice) and ddH2O (three times). 500 μL FC-40 (Sigma) was added in the fluorophilic 24 well plate to form the bottom liquid layer, 2 mL of MSC growth medium was directly added on the FC-40 layer, 5K cells were seeded in each well.

### REFERENCES

1 Di Cio, S. & Gautrot, J. E. Cell sensing of physical properties at the nanoscale: mechanisms and control of cell adhesion and phenotype. Acta Biomater 30, 26-48 (2016).
2 Discher, D. E., Mooney, D. J. & Zandstra, P. W. Growth factors, matrices, and forces combine and control stem cells. Science 324, 1673-1677 (2009).
3 Guilak, F. et al. Control of stem cell fate by physical interactions with the extracellular matrix. Cell Stem Cell 5, 17-26 (2009).
4 Levental, K. R. et al. Matrix crosslinking forces tumor progression by enhancing integrin signalling. Cell 139, 891-906 (2009).
5 Parsons, J. T., Horwitz, A. R. & Schwartz, M. A. Cell adhesion: integrating cytoskeletal dynamics and cellular tension. Nat. Rev. 11, 633-643 (2010).
6 Trappmann, B. et al. Extracellular matrix tethering regulates stem cell fate. Nat. Mater. 11, 642-649 (2012).
7 Murrell, M., Kamm, R. & Matsudaira, P. Substrate viscosity enhances correlation in epithelial sheet movement. Biophys. J. 101, 297-306 (2011).
8 Chaudhuri, O. et al. Substrate stress relaxation regulates cell spreading. Nat. Commun. 6, 6364 (2015).
9 Giaever, I. & Keese, C. R. Behavior of cells at fluid interfaces. Proc. Natl. Acad. Sci. 80, 219-222 (1983).
10 Keese, C. R. & Giaever, I. Cell growth on liquid microcarriers. Science 219, 1448-1449 (1983).
11 Keese, C. R. & Giaever, I. Cell growth on liquid interfaces: Role of surface active compounds. Proc. Natl. Acad. Sci. 80, 5622-5626 (1983).
12 Fuller, G. G. & Vermant, J. Complex fluid-fluid interfaces: rheology and structure. Annu. Rev. Chem. Biomol. Eng. 3, 519-543 (2012).
13 Bos, M. A. & van Vliet, T. Interfacial rheological properties of adsorbed protein layers and surfactants: a review. Adv. Colloid. Interf. Sci. 91, 437-471 (2001).
14 Baldursdottir, S. G., Fullerton, M. S., Nielsen, S. H. & Jorgensen, L. Adsorption of proteins at the oil/water interface—Observation of protein adsorption by interfacial shear stress measurements. Colloid Surf. B Biointerfaces 79, 41-46 (2010).
15 Freer, E. M., Yim, K. S., Fuller, G. G. & Radke, C. J. Interfacial rheology of globular and flexible proteins at the hexadecane/water interface: comparison of shear and dilatation deformation. J. Phys. Chem. B 108, 3835-3844 (2004).
16 Campana, M. et al. Adsorption of bovine serum albumin (BSA) at the oil/water interface: a neutron reflection study. Langmuir 31, 5614-5622 (2015).
17 Wilde, P. J. & Clark, D. C. The competitive displacement of b-lactoglobulin by tween 20 from oil-water and air-water interfaces. J. Colloid Interface Sci. 155, 48-54 (1993).
18 Bellamy, L. J. The infrared spectra of complex molecules. (Wiley, 1959).
19 McClellan, S. J. & Franses, E. I. Adsorption of bovine serum albumin at solid/aqueous interfaces. Colloid. Surf. A: Physicochem. Eng. Aspects 260, 265-275 (2005).
20 Gardel, M. L. et al. Traction stress in focal adhesions correlates biphasically with actin retrograde flow speed. J. Cell Biol. 183, 999-1005 (2008).
21 Novak, P. et al. Nanoscale live-cell imaging using hopping probe ion conductance microscopy. Nat. Mater. 6, 279-281 (2009).
22 Harris, A. R., Daeden, A. & Charras, G. T. Formation of adherens junctions leads to the emergence of a tissue-level tension in epithelial monolayers. J. Cell Sci. 127, 2507-2517 (2014).
23 Gautrot, J. E. et al. Mimicking normal tissue architecture and perturbation in cancer with engineered micro-epidermis. Biomaterials 33, 5221-5229 (2012).
24 Chapman, S., Liu, X., Meyers, C., Schlegel, R. & McBride, A. A. Human keratinocytes are efficiently immortalized by a Rho kinase inhibitor. J. Clin. Invest. 120, 2619-2626 (2010).
25 Jones, P. H. & Watt, F. M. Separation of human epidermal stem cells from transit amplifying cells on the basis of differences in integrin function and expression. Cell 73, 713-724 (1993).
26 Huebner, A. et al. Microdroplets: A sea of applications? Lab Chip 8, 1244-1254 (2008).
27 Hanga, M. P.; Murasiewicz, H.; Pacek, A. W.; Nienow, A. W.; Coopman, K.; Hewitt, C. J., Expansion of bone marrow-derived human mesenchymal stem/stromal cells (hMSCs) using a two-phase liquid/liquid system. J. Chem. Technol. Biotechnol. 2017, 92, 1577-1589.

1-76. (canceled)

77. A method of culturing adherent stem cells at a liquid-liquid interface in a cell culture system, the cell culture system comprising:
a. an aqueous cell culture medium; and
b. an oil phase, wherein the oil phase is functionalised with a conditioning layer that is disposed between the aqueous cell culture medium and the oil phase and the conditioning layer comprises a surfactant and a protein or peptide layer;
the method comprising culturing the adherent cells in the cell culture system at the interface between the oil phase and the aqueous cell culture medium wherein:
i) the oil phase comprises a non-fluorinated oil; and
ii) the surfactant is a non-fluorinated surfactant.

78. The method of claim 77, wherein the surfactant is bonded to the protein or peptide layer via covalent or supramolecular bonds, optionally wherein the surfactant is a non-polymeric surfactant, or the surfactant is a polymeric surfactant.

79. The method of claim 77, wherein the conditioning layer further comprises:
a) a non-peptidic polymer layer disposed between the oil phase and the protein or peptide layer, and wherein the non-peptidic polymer layer comprises the surfactant; or
b) one or more additional polymer layers disposed between the surfactant and the protein or peptide layer, optionally wherein the one or more additional polymer layers are non-peptidic polymer layers, optionally wherein the conditioning layer further comprises at least two different additional polymers layers, or at least 4 polymer layers, or wherein the conditioning layer comprises at least 4 layers of two different polymers arranged in alternating layers.

80. The method of claim 77, wherein the method is for the long-term culture of stem cells, optionally wherein the stem cells are cultured for at least about 1 day, or at least about 5 days, or at least about 7 days, or at least about 14 days, optionally wherein at least 80% of the cells are alive at the end of the culture period.

81. The method of claim 77, wherein the elasticity of the liquid-liquid interface is sufficient for the cells to proliferate to at least about 50% confluency.

82. The method of claim 77, wherein the surfactant
a) comprises a reactive group,
optionally wherein the surfactant comprises a reactive group that allows the formation of covalent or supramolecular bonds between the surfactant and the conditioning layer, or between the surfactant and the protein in the conditioning layer or between the surfactant and a non-protein polymer in the conditioning layer,
optionally wherein the group that allows the formation of covalent or supramolecular bonds is selected from the group consisting of activated carboxylic acids, activated carbonates, azides, alkenes, alkynes, alkoxysilanes, ketoximes, acetoxysilanes, biotin, streptavidin, cyclodextrin, cucurbituril, cyclobis(paraquat-p-phenylene), sequences of nucleic acid molecules, self-aggregating or self-assembling peptides, and peptides enabling specific binding to other molecules; and/or
b) is an acyl chloride surfactant,
optionally wherein the surfactant is selected from the group consisting of octanoyl chloride, sebacoyl chloride and or heptadecanoyl chloride, or a combination thereof,
optionally wherein the surfactant is present in the cell culture system at a concentration of from about 0.001 mg/ml to about 0.05 mg/ml, or from about 0.00125 mg/ml to about 0.05 mg/ml, or from about 0.00125 mg/ml to about 0.01 mg/ml.

83. The method of claim 77, wherein the non-fluorinated oil is selected from the group consisting of silicone oil, optionally wherein the silicone oil is polydimethylsiloxane (PDMS) or an associated derivative, paraffin oil, mineral oil, fatty acid oils, castor oil, palm oil, rapeseed oil and olive oil.

84. The method of claim 77, wherein:
a. the surfactant is octanoyl chloride and the oil is polydimethylsiloxane;
b. the surfactant is sebacoyl chloride and the oil is polydimethylsiloxane;
c. the surfactant is heptadecanoyl chloride and the oil is polydimethylsiloxane;
d. the surfactant is a combination of heptadecanoyl chloride and sebacoyl chloride and the oil is polydimethylsiloxane;
e. the surfactant is heptadecanoyl chloride and the oil is rapeseed oil;
f. the surfactant is a combination of heptadecanoyl chloride and sebacoyl chloride and the oil is rapeseed oil; or
g. the surfactant is a combination of heptadecanoyl chloride and sebacoyl chloride and the oil is mineral oil.

85. The method of claim 77, wherein the conditioning layer comprises a polymer selected from the group consisting of poly(L)-lysine (PLL), poly(allylamine), poly(vinyl alcohol) (PVA), poly(hydroxyethyl methacrylate) (PHEMA), chitosan, poly(serine), dextran, heparin, poly(styrene sulfonate), chondroitin sulfate, hyaluronic acid, carboxy methyl cellulose, albumin, lysozyme, lactoglobulin, fibronectin, collagen, laminin, agrin, fibroin, elastin, elastin like proteins (ELPs), resilin, sericin, xanthan gum, alginate, gelatine, poly(sulfopropyl methacrylate), poly(acrylic acid), poly(methacrylic acid), gantrez, poly(maleic acid-alt-styrene) and a composite of PLL and graphene oxide,
optionally wherein the polymer is positively charged,
optionally, wherein the polymer comprises a reactive group,
optionally wherein the polymer comprises a reactive group that allows the formation of covalent or supramolecular bonds between the polymer and the conditioning layer or between the polymer and a protein in the conditioning layer or between the polymer and the surfactant in the conditioning layer,
optionally wherein the reactive group is selected from the group consisting of amine, alcohol or thiol groups.

86. The method of claim 77, wherein the protein or peptide layer comprises an extra-cellular matrix (ECM) protein or macromolecule mimicking the cell adhesive properties of ECM proteins, optionally wherein the protein is selected from the group consisting of fibronectin, laminin, collagen, vitronectin, agrin, fibroin and elastin, or fragments thereof,
or wherein the protein or macromolecule mimicking the cell adhesive properties of ECM proteins comprises a cell adhesive peptide, optionally wherein the cell adhesive peptide sequence comprises a peptide sequence selected from the group consisting of RGD, YIGSR, IKVAV and PHSRN.

87. The method of claim 77, wherein the protein is crosslinked after assembly at the oil-water interface.

88. The method of claim 77, wherein the cell culture system comprises:
a. an oil selected from the group consisting of a rapeseed oil, mineral oil and PDMS;
b. a surfactant selected from the group consisting of octanoyl chloride, sebacoyl chloride and heptadecanoyl chloride, or a combination thereof;
c. a polymer selected from the group consisting of PLL, poly(sodium 4-styrenesulfonate), ELP and hyaluronic acid; and
d. a protein selected from the group consisting of collagen and fibronectin, or a combination thereof, or
wherein the cell culture system comprises:
e. PLL and fibronectin;
f. PDMS, octanoyl chloride, PLL and fibronectin;
g. PDMS, octanoyl chloride, PLL, poly(sodium 4-styrenesulfonate) and collagen type I;
h. PDMS, sebacoyl chloride, PLL and fibronectin;
i. PDMS, sebacoyl chloride, PLL, poly(sodium 4-styrenesulfonate) and collagen;
j. PDMS, heptadecanoyl chloride, PLL and fibronectin;
k. PDMS, heptadecanoyl chloride, sebacoyl chloride, PLL and fibronectin;
l. Rapeseed oil, heptadecanoyl chloride, PLL and fibronectin;
m. Rapeseed oil, heptadecanoyl chloride, sebacoyl chloride, PLL and fibronectin; or
n. Mineral oil, heptadecanoyl chloride, sebacoyl chloride, PLL and fibronectin.

89. The method of claim 77, wherein the cells are selected from a group consisting of human primary keratinocytes (HPKs), mesenchymal stem cells (MSCs), induced pluripotent stem cells (iPSCs), adipose derived stem cells amniotic fluid derived stem cells, and cord blood stem cells,
optionally wherein the cell culture system is in the form of an emulsion or in the form of a planar sheet.

90. The method of claim 77, wherein the elasticity of the conditioning layer is at least about 60%, and/or the shear interfacial modulus of the interface between the aqueous medium and the oil phase is at least about 0.01 N/m, and/or the pH of the cell conditioning layer is from about 9 to about 11.

91. A culture system for the culture of adherent stem cells at a liquid-liquid interface, the culture medium comprising:
a. an aqueous cell culture medium; and
b. an oil phase, wherein the oil phase is functionalised with a conditioning layer that is disposed between the aqueous cell culture medium and the oil phase and the conditioning layer comprises a surfactant and a protein or peptide layer, wherein the oil phase comprises a non-fluorinated oil; and the surfactant is a non-fluorinated surfactant.

92. The culture system of claim 91, wherein the surfactant is bonded to the protein or peptide layer via covalent or supramolecular bonds, optionally wherein the surfactant is a non-polymeric surfactant, or the one or more surfactants are polymeric surfactants.

93. A method of expanding a population of adherent cells comprising culturing the cells at a liquid-liquid interface according to the method of claim 77 and harvesting the cells from the culture medium.

94. The method according to claim 93 wherein the adherent cells are cultured to at least 50% confluence prior to harvesting.

95. The culture system of claim 91, wherein the culture system is contained in a bioreactor comprising a culture of stem cells, wherein the stem cells adhere to a liquid-liquid interface in the cell culture medium.

96. The bioreactor of claim 95, wherein the stem cells are at 50% confluence or above, optionally wherein the bioreactor is a cell culture flask or bag.

97. The method of claim 89, wherein the cells are selected from a group consisting of human primary keratinocytes (HPKs), mesenchymal stem cells (MSCs), induced pluripotent stem cells (iPSCs), adipose derived stem cells, amniotic fluid derived stem cells, cord blood stem cells.
